(19) United States
(12) Patent Application Publication
(10) Pub. No.: US 2012/0296189 A1
(43) Pub. Date: Nov. 22, 2012

(54) Methods of Collecting and Analyzing Samples

(75) Inventors: John Bhogal, San Francisco, CA (US); Shridhara Alva Karinka, Pleasanton, CA (US); Timothy P. Henning, Vernon Hills, IL (US); David Cunningham, Lake Villa, IL (US); Udo Hoss, Castro Valley, CA (US); Andrew H. Naegeli, Walnut Creek, CA (US); John LaTour, Alameda, CA (US)

(73) Assignee: Abbott Diabetes Care Inc.

(21) Appl. No.: 13/458,556

(22) Filed: Apr. 27, 2012

Related U.S. Application Data
(60) Provisional application No. 61/480,883, filed on Apr. 29, 2011.

(51) Int. Cl. G01N 30/96; G01N 33/00; A61B 5/00

(52) U.S. Cl. 600/365; 600/573; 600/309; 73/61.43; 73/61.52

(57) ABSTRACT

Provided are methods of measuring analyte concentrations in interstitial fluid samples, methods of determining accuracy of subcutaneously implantable analyte sensors, methods of manufacturing and determining calibration factors for subcutaneously implantable analyte sensors, as well as subcutaneously implantable analyte sensors manufactured according to the described methods and having a high level of accuracy. Methods of determining the concentration of an analyte in a bodily sample, and methods of extracting interstitial fluid are also provided.

### CROSS-REFERENCE TO RELATED APPLICATIONS

Pursuant to 35 U.S.C. §119(e), this application claims priority to U.S. Provisional Patent Application No. 61/480,883 filed on Apr. 29, 2011, the disclosure of which is herein incorporated by reference in its entirety.

### INTRODUCTION

In many instances it is desirable or necessary to regularly monitor the concentration of particular constituents in a fluid. A number of systems are available that analyze the constituents of bodily fluids such as blood, urine and saliva. Examples of such systems conveniently monitor the level of particular medically significant fluid constituents, such as, for example, cholesterol, ketones, vitamins, proteins, and various metabolites or blood sugars, such as glucose. Diagnosis and management of patients suffering from diabetes mellitus, a disorder of the pancreas where insufficient production of insulin prevents normal regulation of blood sugar levels, requires carefully monitoring of blood glucose levels on a daily basis. A number of systems that allow individuals to easily monitor their blood glucose are currently available. Such systems include electrochemical biosensors, including those that comprise a glucose sensor that is adapted for insertion into a subcutaneous site within the body for the continuous monitoring of glucose levels in bodily fluid of the subcutaneous site (see for example, U.S. Pat. No. 6,175,752 to Say et al).

A person may obtain a blood sample by withdrawing blood from a blood source in his or her body, such as a vein, using a needle and syringe, for example, or by lancing a portion of his or her skin, using a lancing device, for example, to make blood available external to the skin, to obtain the necessary sample volume for in vitro testing. The person may then apply the fresh blood sample to a test strip, whereupon suitable detection methods, such as calorimetric, electrochemical, or photometric detection methods, for example, may be used to determine the person's actual blood glucose level. The foregoing procedure provides a blood glucose concentration for a particular or discrete point in time, and thus, must be repeated periodically, in order to monitor blood glucose over a longer period.

In addition to the discrete or periodic, or in vitro, blood glucose-monitoring systems described above, at least partially implantable, or in vivo, blood glucose-monitoring systems, which are constructed to provide continuous in vivo measurement of an individual's blood glucose concentration, have been described and developed. Such analyte monitoring devices are constructed to provide for continuous or automatic monitoring of analytes, such as glucose, in the blood stream or interstitial fluid. Such devices include electrochemical sensors, at least a portion of which are operably positioned in a blood vessel or in the subcutaneous tissue of a user.

In the case of in vivo analyte monitoring systems designed to determine analyte concentration in interstitial fluid (ISF), determining the accuracy of such systems has traditionally involved comparing the analyte concentration obtained using the system to an analyte concentration obtained from a reference blood sample, e.g., using a disposable test strip and meter to determine a reference blood analyte concentration. However, a time lag often exists between the interstitial fluid analyte concentration and the blood analyte concentration. For example, a time lag in the distribution of glucose from blood to the interstitium has been observed. As a result of this lag, blood glucose concentrations do not correlate precisely with interstitial glucose concentrations. It follows that assessing the accuracy of an in vivo glucose monitoring system by comparing the ISF glucose concentration measured by the system to a reference blood glucose concentration is inherently flawed. Accordingly, there is a need for alternative approaches for determining the accuracy of in vivo analyte monitoring systems. The present disclosure provides such approaches.

### SUMMARY

Provided are methods of measuring analyte concentrations in interstitial fluid samples, methods of determining accuracy of subcutaneously implantable analyte sensors, methods of manufacturing and determining calibration factors for subcutaneously implantable analyte sensors, as well as subcutaneously implantable analyte sensors manufactured according to the described methods and having a high level of accuracy. Methods of determining the concentration of an analyte in a bodily sample, and methods of extracting interstitial fluid are also provided.

### BRIEF DESCRIPTION OF THE FIGURES

FIGS. 1A-1E show an embodiment of a rotary abrading device according to the embodiments of the present disclosure.

FIGS. 2A-F show an embodiment of the methods of the present disclosure that includes abrading an epidermal skin layer, applying a vacuum pressure, and collecting presented interstitial fluid sample from a subject.

FIG. 3 shows ion chromatography results of an example sugar test mixture.

FIG. 4 shows ion chromatography results of a NERL D-glucose standard.

FIG. 5 shows ion chromatography results of a human interstitial fluid sample obtained using one embodiment of the present disclosure.

FIG. 6 shows a Clarke Error Grid for assessing the accuracy of in vivo interstitial fluid glucose sensors.

FIG. 7 shows a block diagram of an embodiment of an analyte monitoring system according to embodiments of the present disclosure.

FIG. 8 shows a block diagram of an embodiment of a data processing unit of the analyte monitoring system shown in FIG. 1.

FIG. 9 shows a block diagram of an embodiment of the primary receiver unit of the analyte monitoring system of FIG. 1.

FIG. 10 shows a schematic diagram of an embodiment of an analyte sensor according to the embodiments of the present disclosure.

FIGS. 11A-11B show a perspective view and a cross sectional view, respectively, of an embodiment of an analyte sensor.

### DETAILED DESCRIPTION

Provided are methods of measuring analyte concentrations in interstitial fluid samples, methods of determining accuracy of subcutaneously implantable analyte sensors, methods of manufacturing and determining calibration factors for subcutaneously implantable analyte sensors, as well as subcutaneously implantable analyte sensors manufactured according to the described methods and having a high level of accuracy. Methods of determining the concentration of an analyte in a bodily sample, and methods of extracting interstitial fluid are also provided.

Before the methods of the present disclosure are described in greater detail, it is to be understood that the methods are not limited to particular embodiments described, as such may, of course, vary. It is also to be understood that the terminology used herein is for the purpose of describing particular embodiments only, and is not intended to be limiting, since the scope of the methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods. The upper and lower limits of these smaller ranges may independently be included in the smaller ranges and are also encompassed within the methods, subject to any specifically excluded limit in the stated range. Where the stated range includes one or both of the limits, ranges excluding either or both of those included limits are also included in the methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods belong. Although any methods and materials similar or equivalent to those described herein can also be used in the practice or testing of the methods, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present methods are not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, which are, for clarity, described in the context of separate embodiments, may also be provided in combination in a single embodiment. Conversely, various features of the methods, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed, to the extent that such combinations embrace operable processes and/or devices/systems/kits. In addition, all sub-combinations listed in the embodiments describing such variables are also specifically embraced by the present methods and are disclosed herein just as if each and every such sub-combination of chemical groups was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order which is logically possible.

### Methods

Disclosed herein are methods of measuring analyte concentrations in interstitial fluid samples, methods of determining accuracy of subcutaneously implanted analyte sensors, and methods of calibrating subcutaneously implanted analyte sensors. Also disclosed are methods of determining the concentration of an analyte in a bodily sample, and methods of extracting interstitial fluid. Detailed descriptions of the methods are provided in turn below.

Methods of Measuring the Concentration of Analytes in Interstitial Fluid Samples

As summarized above, certain embodiments are directed to methods of measuring interstitial fluid analyte concentrations. According to one embodiment, a method of measuring the concentration of an analyte in an interstitial fluid sample is provided. An interstitial fluid sample may be collected from a subject using any one of a variety of different methods and devices. Exemplary methods of forming an opening in a portion of a subject's skin to provide access to interstitial fluid include, but are not limited to, use of one or more microneedles (e.g. a microneedle array), a microporator, a rotating lancet, a rotating drill, a laser, a thermal ablation device, and the like. Other exemplary methods for collecting interstitial fluid from a subject include, but are not limited to, use of an ultrasound energy system (e.g., a low-frequency ultrasound energy system) such as the Sontra SonoPrep® ultrasonic skin permeation system (Sontra Medical Corp., Franklin, Mass.), a hydrogel-containing device (e.g., see WO 1996/000110, WO 1997/002811, US 2011/0034787, the disclosures of which are incorporated herein by reference in their entirety for all purposes), microfluidic-based transdermal interstitial fluid collection devices (e.g., see Paranjape, M., et al., (2003) Sensors and Actuators A: Physical 104(3): 195-204), and the like. Exemplary methods and devices are described in U.S. Provisional Patent Application Ser. No. 61/481,125 entitled “Devices and Methods for Obtaining Analyte Sample” filed on Apr. 29, 2011, and in U.S. Pat. No. 6,155,992, the disclosures of which are incorporated herein by reference in their entirety for all purposes. Any of the above approaches for collecting interstitial fluid from a subject may be employed, alone or in combination, when practicing any of the methods of the present disclosure, e.g., methods of measuring the concentration of an analyte in an interstitial fluid sample, methods of determining accuracy of a subcutaneously implanted analyte sensor, methods of calibrating a subcutaneously implanted analyte sensor, methods of determining the concentration of an analyte in a sample, and so forth.

One non-limiting embodiment of collecting an interstitial fluid sample includes abrading the skin to provide access to the interstitial fluid. Such an exemplary method includes abrading an epidermal skin layer of a subject (e.g., a human subject) at a first site, applying a vacuum to the abraded epidermal skin layer such that interstitial fluid presents on a surface of the abraded epidermal skin layer, and collecting the presented interstitial fluid to obtain the interstitial fluid sample.

Abrading an epidermal skin layer of a subject can be carried out using any suitable abrading means. In one embodiment, the abrading is performed using a rotary device. Rotary devices are commercially available, such as the Dremel® and Roto-Zip® rotary hand tools sold by the Robert Bosch Tool Corporation (Mount Prospect, Ill.). An end of the rotary device may be configured to connect to various accessories or attachments. In one embodiment, the abrading is performed using a rotary device, and the device includes a rotatable tip, e.g., a rotatable grindstone. The rotatable grindstone is optionally removable, such that after the epidermal skin layer of a subject is abraded, the grindstone can be removed, sterilized, and reused to, e.g., abrade an epidermal skin layer of the same or a different subject.

While practicing the subject methods of measuring the concentration of an analyte in an interstitial fluid sample, it is desirable that the abrading does not cause blood to present on the surface of the abraded epidermal skin layer. One approach for preventing blood from forming at the surface of the abraded epidermal skin layer is to control the depth of the abrasion, e.g., by physically preventing the tip from abrading beyond a predefined depth of the epidermis. As such, in one embodiment, the abrading is performed using a rotary device that includes a rotatable grindstone, where the rotary device also includes a housing that prevents the rotatable grindstone from abrading the skin at a depth where blood vessels reside (e.g., the housing prevents the rotating grindstone from disrupting, e.g., abrading, a blood vessel, such as a capillary, beneath the epidermal skin layer). The housing may be attached to the rotary device in such a way that only a defined portion, e.g., a defined length, of the rotatable tip emerges from the housing and is able to abrade the epidermal skin layer when the housing is placed on the skin directly or indirectly (e.g., via a frame attached to the skin and configured to receive the housing).

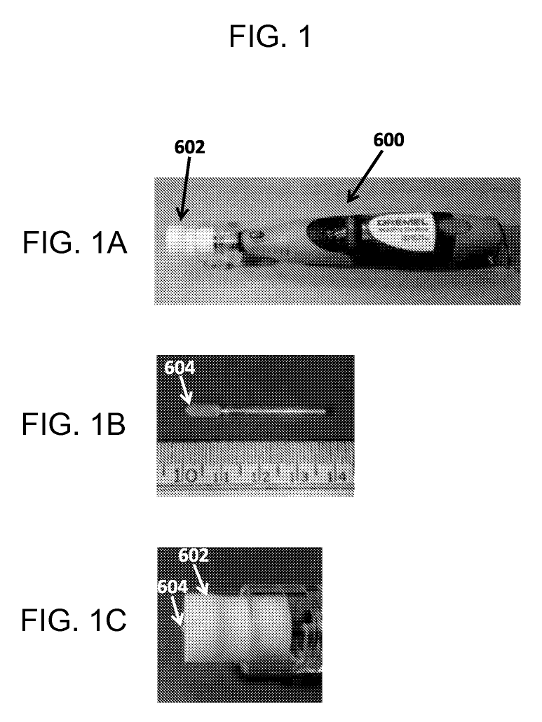

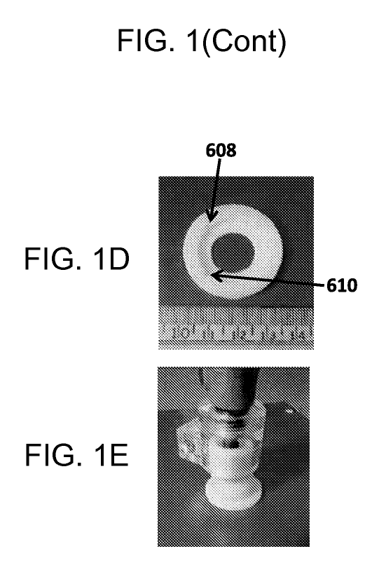

A rotary device configured to abrade an epidermal skin layer of a subject according to one embodiment is shown in FIG. 1. As shown in FIG. 1A, rotary device 600 includes housing 602 that may be placed on the skin of a subject (either directly or indirectly) during the abrading step, where the housing prevents the abrading from occurring at a depth of the skin where blood vessels (e.g., capillaries) reside. Here, the rotary device is configured to receive a bit having rotatable grindstone tip 604 (shown at FIG. 1B). FIG. 1C shows the abrading end of rotary device 600, where a defined portion of rotatable grindstone 604 is exposed beyond a cross-sectional plane at the distal end of housing 602.

When the rotary device includes a housing (e.g., such as housing 602 in FIG. 1), the housing can be mounted on a frame placed upon the skin of the subject, e.g., a frame attached to (e.g., adhered to) the outer surface of the skin. The frame may have notches complementary to features of the housing, so that the orientation of the housing (and accordingly, the rotatable tip) relative to the frame can be precisely controlled. Such a frame may be helpful when it is desirable to abrade multiple sites at defined distances from each other. For example, the rotatable tip may be offset (not precisely in the center) relative to the housing, and rotating the housing within the frame at defined positions facilitates abrasion at two or more defined sites within the area of the skin encompassed by the frame. Accordingly, in one embodiment, a frame, e.g., skin stabilizing frame 606 as shown in FIG. 1D, may be used to further control the location and/or depth of the abrading. As shown, skin stabilizing frame 606 has one or more notches, e.g., notches 608 and 610 visible in FIG. 1D, complementary to features of the housing. FIG. 1E shows the abrading end of the rotary device, where the housing is reversibly coupled to skin stabilizing frame 606.

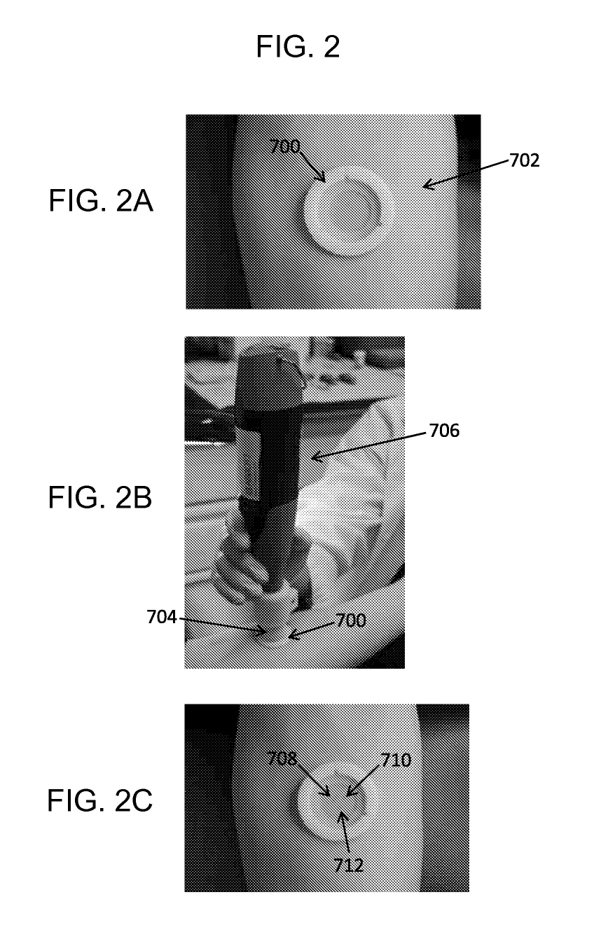

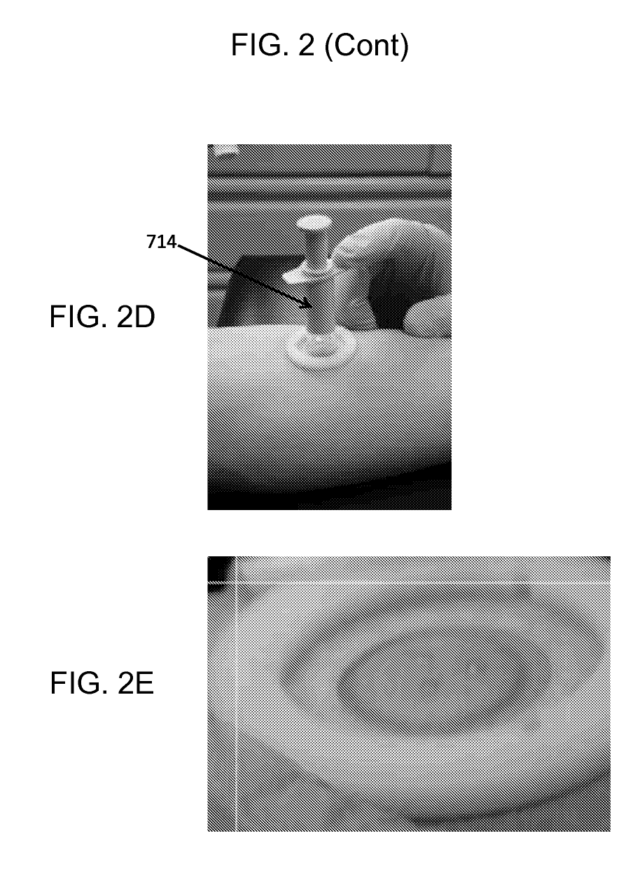

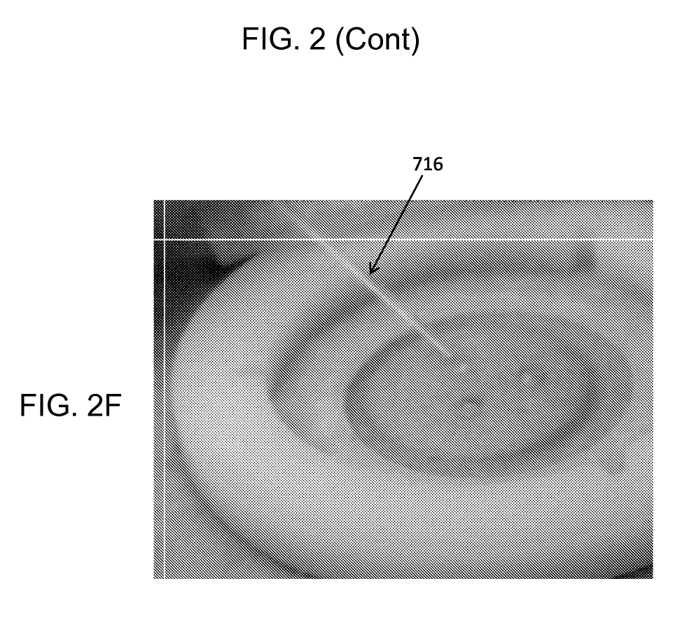

Use of a rotary device on a subject in accordance with one embodiment of the present disclosure is shown in FIG. 2. FIG. 2A shows skin stabilizing frame 700 affixed to forearm 702 of the subject. As shown in FIG. 2B, rotary device 706 includes rotatable tip (not shown) and housing 704 that reversibly couples to skin stabilizing frame 700. Once the rotary device is coupled to the skin stabilizing frame, the abrading of an epidermal layer of the subject may commence for a suitable period of time at a suitable speed (e.g., rpm). FIG. 2C shows the forearm of the subject subsequent to abrasion at three defined sites (sites 708-712).

When the subject methods employ a rotary device with a rotatable tip (e.g., a rotatable grindstone) to abrade an epidermal skin layer of a subject, the abrading may be performed at any suitable speed for any suitable time. For example, the abrading may be performed at speeds ranging from about 2,000 to 12,000 revolutions per minute (rpm), for example, from about 4,000 to 10,000 rpm, such as from about 5,000 to 8,000 rpm, including from about 5,500 to 7,500 rpm. The abrading may be performed for durations ranging from, e.g., about 10 seconds to 2 minutes, for example, from about 15 seconds to 1 minute, such as from about 20 seconds to 50 seconds, including from about 30 seconds to 45 seconds.

When practicing the subject methods, abrading an epidermal skin layer includes abrading the epidermal skin layer at any suitable depth, e.g., a depth that permits the presentation of interstitial fluid (but preferably not blood) on the surface of the abraded skin. In one embodiment, the abrading includes abrading the epidermal skin layer at a depth ranging from about 0.5 to 3 millimeters (mm), such as from about 1 to 2.5 mm, including from about 1.5 to 2 mm. In a related embodiment, the abrading can include abrading a portion or the entire depth of the stratum corneum at one or more sites on the subject. The stratum corneum is an approximately 10-40 μm thick outermost skin layer comprised of dead skin cells. The stratum corneum constitutes the main barrier preventing interstitial fluid from forming droplets on the surface of the skin.

In accordance with the subject methods, the abrading may be performed at any location on the subject's body suitable for obtaining an interstitial fluid sample. For example, the abrading may be performed on the subject's arm (e.g., a forearm or upper arm), leg (e.g., a lower leg or upper leg), thorax (e.g., chest), abdomen, a region at or near the boundary of the thorax and abdomen, back, and so forth. In one embodiment, the epidermal skin layer is abraded at a first site located at an area of the subject selected from a forearm, an upper arm, and an abdomen.

The abrading may further include abrading an epidermal skin layer of the subject at a second site (e.g., as in the embodiment shown in FIG. 2). In one embodiment, the second site is adjacent to the first site. For example, the second site may be located at a distance from the first site ranging from about 2 to 40 mm, such as from about 3 to 20 mm, including from about 4 to 10 mm (e.g., from about 5 to 7.5 mm). In another embodiment, the second site is contiguous with the first site, e.g., the two sites may overlap to create a “trough” from which interstitial fluid is subsequently collected. The epidermis of the subject may be abraded at any number of sites suitable for collecting the desired amount of interstitial fluid. For example, 1 or more, 2 or more, 3 or more, 4 or more, 5 or more, or 6 or more sites (e.g., 10 or more sites) on the epidermis of the subject may be abraded in accordance with the subject methods.

As noted above, the methods include applying a vacuum to the abraded epidermal skin layer such that interstitial fluid presents on a surface of the abraded epidermal skin layer. Any suitable vacuum device capable of facilitating the presentation of interstitial fluid on the surface of the abraded epidermal skin layer may be used, e.g., the EZ-VAC™ vacuum device available from AmbiMedlnc (Scotts Valley, Calif.), an equivalent thereof, and so forth.

Depending on a number of factors, accurately measuring the concentration of the analyte in the interstitial fluid sample may be difficult when blood is present in the interstitial fluid sample. As noted above, a first approach to preventing blood contamination of the interstitial fluid sample is to prevent blood vessel disruption during the abrading step. Similarly, the step of applying a vacuum to the abraded epidermal skin layer may be performed in a manner that prevents the disruption of blood vessels and the appearance of blood on the abraded skin surface. In one embodiment, applying a vacuum includes applying a vacuum pressure that does not cause blood to present on the abraded epidermal skin layer. For example, the vacuum pressure may be controlled such that it does not exceed a critical pressure beyond which the walls of blood vessels beneath the epidermal layer are likely to be damaged and/or leak. The vacuum pressure may range from, e.g., about 2 to 40 inches of mercury, such as about 4 to 30 inches of mercury, including about 5 to 25 inches of mercury. For example, applying a vacuum optionally comprises applying a vacuum pressure ranging from about 6 to 20 inches of mercury.

Applying a vacuum to the abraded epidermal skin layer in accordance with one embodiment of the present disclosure is shown in FIG. 2D. In this example, vacuum device 714 has a substantially cylindrical skin contacting end that surrounds the one or more abrasion sites and forms a seal with the skin sufficient to permit the application of a suitable vacuum pressure to the abrasion sites. The presentation (or “expression”) of interstitial fluid at sites 708-712 is shown at FIG. 2E.

To obtain the interstitial fluid sample for subsequent analyte measurement, the presented (or “expressed”) interstitial fluid may be collected by any suitable collection means. For example, the collecting is optionally performed using one or more micro-capillary collection tubes. Micro-capillary collection tubes, such as the Drummond MICROCAPS™ from Drummond Scientific Co. (Broomall, Pa.) are commercially available. Other approaches for collecting the presented interstitial fluid are possible, e.g., using a micropipettor with appropriately sized disposable tip to draw the interstitial fluid into the tip for subsequent expulsion into a suitable storage tube. FIG. 2F shows the collection of presented interstitial fluid using micro-capillary tube 716.

Once the interstitial fluid samples are collected, the samples may be frozen (e.g., to prevent degradation of the analyte) and stored indefinitely. Optionally, the subject methods further include diluting the interstitial fluid sample prior to measuring the concentration of the analyte. For example, if the sample will be subjected to ion chromatography in order to determine the concentration of the analyte, each sample may be added to an aqueous benzoic acid solution (e.g., 0.5 μL of interstitial fluid sample in 250 μL of aqueous benzoic acid solution) and immediately frozen until just prior to analysis by ion chromatography.

The analyte to be measured may be any analyte of interest, including but not limited to acetyl choline, amylase, bilirubin, cholesterol, chorionic gonadotropin, glycosylated hemoglobin (HbA1c), creatine kinase (e.g., CK-MB), creatine, creatinine, DNA, fructosamine, glucose, glucose derivatives, glutamine, growth hormones, hormones, ketones, ketone bodies, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In embodiments that monitor more than one analyte, the analytes may be monitored at the same or different times. In one embodiment, the analyte is glucose.

Measuring the concentration of the analyte in the interstitial fluid sample can be carried out using any approach suitable for the analyte of interest, including but not limited to chromatography (e.g., ion chromatography, HPLC), a test strip and corresponding meter capable of measuring analyte levels in interstitial fluid, quantitative electrophoresis-based assays (e.g., where the target analyte is labeled (e.g., radiolabeled, fluorescently-labeled, etc.), quantitative spectroscopic-based assays, mass spectrometry, quantitative colorimetric assays, noninvasive approaches (e.g., using Raman spectrometers, or any other electromagnetic radiation-based devices configured to non-invasively determine the concentration of an analyte in blood, interstitial fluid, or other bodily fluid or tissue), combinations thereof, etc. For example, a chromatographic assay may be used to determine the analyte concentration in the sample. In one embodiment, the analyte concentration is measured by high-performance liquid chromatography (HPLC). In a related embodiment, the analyte concentration is measured by ion chromatography (IC, or “ion-exchange chromatography”). Ion chromatography systems are known in the art and commercially available from, e.g., Dionex Corporation (Sunnyvale, Calif.). Detailed guidance and protocols for detecting and measuring the concentration of analytes of interest by ion chromatography can be found, e.g., in Fritz and Gjerde, “Ion Chromatography”, 4th edition, Apr. 14, 2009, and Weiss and Weiss, “Handbook of Ion Chromatography”, 3rd edition, Jan. 3, 2005.

In certain aspects of the present disclosure, the analyte is glucose, and the glucose concentration is measured using ion chromatography. Glucose standards (e.g., NERL D-Glucose standards) at varying concentrations may be used. The standards and interstitial fluid samples are diluted similarly prior to ion chromatography, and the glucose concentrations in the samples may be determined by comparison with the glucose standards of known concentration. For example, NERL D-Glucose standards of 50 mg/dL, 100 mg/dL, 200 mg/dL and 400 mg/dL can be used to make standard solutions having glucose concentrations of 100 μg/dL, 200 μg/dL, 400 μg/dL and 800 μg/dL, respectively. Each standard solution may be injected on the column (e.g., a Dionex CarboPac PA10, 2 mm diameter column) in order to make a calibration curve. The interstitial fluid samples may be appropriately diluted and injected on the column. The interstitial fluid samples may be injected on the column more than once, and the average of the sample injections may be used for a single reportable value. The peak area of the standard and sample can be determined from their respective chromatograms. The calibration curve may be determined by linear regression (peak area vs. standard concentration). The glucose concentration of each sample can be measured based on the calibration curve. Glucose eluted from the column may be detected using any suitable detector, e.g., an ED electrochemical detector from Dionex Corp. (Sunnyvale, Calif.). A detailed protocol for determining the glucose concentration in an interstitial fluid sample according to one embodiment of the present disclosure is provided in the Experimental section (Example 2) below.

In addition to measuring the concentration of the analyte of interest, it may be desirable under certain circumstances to determine the concentration of a secondary analyte. When the concentration of the secondary analyte in interstitial fluid is substantially constant, determining the concentration of the secondary analyte in the interstitial fluid sample could serve as a reference concentration to control for, e.g., potential evaporation of water (and corresponding concentration of the analyte) during application of the vacuum, and any variability in sample handling. As such, the subject methods may further include normalizing the analyte concentration to the concentration of a reference analyte in the interstitial fluid sample.

Any secondary analyte that is present in interstitial fluid at a substantially constant concentration may be used as the reference analyte. The concentrations of physiological salts, for example, are substantially constant in the interstitial fluid. As such, in certain aspects, the methods include normalizing the analyte concentration to the concentration of a physiological salt component in the interstitial fluid sample. The physiological salt component is optionally selected from sodium, potassium, magnesium, calcium, chloride, hydrogen phosphate, and hydrogen carbonate. By way of example, the concentration of the analyte of interest (e.g., glucose) may be measured in the interstitial fluid sample, e.g., by ion chromatography, and the sodium concentration in the same sample can be determined, e.g., by ion chromatography or any other convenient and reliable means. Any disparity between the known (“constant”) concentration of sodium in interstitial fluid and the measured sodium concentration of the interstitial fluid sample may be used to calibrate the concentration measurement for glucose. For example, if the measured sodium concentration is greater than the known concentration of sodium in interstitial fluid by a factor of 2, the measured glucose concentration can be divided by 2 to obtain a normalized glucose concentration that accurately indicates the glucose concentration in the interstitial fluid of the subject.

Methods of Determining the Accuracy of Subcutaneously Implanted Analyte Sensors

As summarized above, methods of determining accuracy of a subcutaneously implanted analyte sensor are provided. The methods include determining an interstitial fluid analyte concentration from a subject using the subcutaneously implanted analyte sensor. The methods also include determining a reference interstitial fluid analyte concentration from the subject, where the determining includes abrading an epidermal skin layer of the subject at a first site, applying a vacuum to the abraded epidermal skin layer such that interstitial fluid presents on a surface of the abraded epidermal skin layer, and measuring the concentration of the analyte in the presented interstitial fluid, e.g., by chromatography (e.g., ion chromatography, HPLC), using a test strip and corresponding meter capable of measuring analyte levels in interstitial fluid, quantitative electrophoresis-based assays (e.g., where the target analyte is labeled (e.g., radiolabeled, fluorescently-labeled, etc.), quantitative spectroscopic-based assays, mass spectrometry, quantitative colorimetric assays, noninvasive approaches (e.g., using Raman spectrometers, or any other electromagnetic radiation-based devices configured to non-invasively determine the concentration of an analyte in blood, interstitial fluid, or other bodily fluid or tissue), combinations thereof, etc. Also included in the methods is comparing the interstitial fluid analyte concentration from the subcutaneously implanted analyte sensor to the reference interstitial fluid analyte concentration, thereby determining the accuracy of the subcutaneously implanted analyte sensor.

As noted above, an initial step in determining the accuracy of a subcutaneously implanted analyte sensor is determining an interstitial fluid analyte concentration from a subject using the subcutaneously implanted analyte sensor. Analyte sensors that find use in the subject methods include, but are not limited to, in vivo analyte sensors that are at least partially implantable within the body of the subject. For example, the analyte sensor may be an electrochemical biosensor adapted for insertion into a subcutaneous site within the body and designed, e.g., for the continuous monitoring of analyte levels in a bodily fluid of the subcutaneous site. Such electrochemical biosensors for the continuous monitoring of, e.g., glucose levels in a subcutaneous bodily fluid are described, e.g., in U.S. Pat. No. 6,175,752 to Say et al., the full disclosure of which is incorporated herein by reference in its entirety for all purposes.

Analyte sensors that find use in the subject methods are described in detail below. Briefly, the sensor may include a first portion positionable above a surface of the skin and a second portion that includes an insertion tip positionable below the surface of the skin, e.g., penetrating through the skin and into, e.g., the subcutaneous space, in contact with a bodily fluid (e.g., interstitial fluid) of the subject. In certain aspects, the sensor operates to electrolyze an analyte of interest in the subcutaneous fluid such that a current is generated between a working electrode and a counter electrode of the sensor. The amount of current generated may correlate to the concentration of the analyte in the interstitial fluid. As such, determining an interstitial fluid analyte concentration from a subject using the subcutaneously implanted analyte sensor may include correlating an electrical signal (e.g., a current) passing through a working electrode to an amount, concentration, or level of an analyte in the interstitial fluid. As described further below, the subcutaneously implanted analyte sensor may be part of an analyte monitoring system that further includes, e.g., a data processing unit connectable to the sensor, a primary receiver unit, and any other system components useful for monitoring and/or managing the analyte concentration in the subject.

The step of determining a reference interstitial fluid analyte concentration from the subject includes the sub-steps of abrading an epidermal skin layer of the subject at a first site, applying a vacuum to the abraded epidermal skin layer such that interstitial fluid presents on a surface of the abraded epidermal skin layer, and measuring the concentration of the analyte in the presented interstitial fluid. Each of these sub-steps is described in detail above in the section entitled “Methods of Measuring the Concentration of Analytes in Interstitial Fluid Samples”. It will be understood that the above description of these sub-steps also describes various embodiments of the methods of determining accuracy of a subcutaneously implanted analyte sensor of the present section.

Embodiments relating to the abrading sub-step are as described above. For example, the abrading may be performed using a rotary device, e.g., a rotary device that includes a rotatable grindstone. The rotary device optionally includes a housing that prevents the rotatable grindstone from abrading a blood vessel, e.g., a capillary beneath the epidermal skin layer. In certain aspects, the abrading includes abrading the epidermal skin layer at a depth ranging from about 0.5 to 3 mm. In other aspects, the abrading includes abrading the stratum corneum. The first site may be located at an area of the subject selected from a forearm, an upper arm, and an abdomen. The abrading may further include abrading an epidermal skin layer of the subject at a second site, e.g., a second site that is adjacent to, or contiguous with, the first site.

Depending on the nature of the analyte, etc., the validity of comparing the concentration measurements from the analyte sensor and the reference interstitial fluid sample may be enhanced by obtaining the reference sample within close proximity to the analyte sensor, e.g., to control for physiological differences between different body regions that may affect the interstitial fluid analyte concentration in a region-specific manner. Accordingly, in one embodiment, the abrading occurs near the location of the subject where the subcutaneous analyte sensor is implanted. For example, if the analyte sensor is subcutaneously implanted in the right forearm of the subject, the abrading may occur on the right forearm of the subject as well (e.g., adjacent to the analyte sensor), and so forth.

Embodiments relating to the sub-step of applying a vacuum are as described above. For example, applying a vacuum pressure optionally includes applying a pressure that does not cause blood to present on the abraded epidermal skin layer. In certain aspects, applying a vacuum to the abraded epidermal skin layer comprises applying a vacuum pressure ranging from about 6 to 20 inches of mercury.

The analyte to be measured may be any analyte of interest, including but not limited to acetyl choline, amylase, bilirubin, cholesterol, chorionic gonadotropin, glycosylated hemoglobin (HbA1c), creatine kinase (e.g., CK-MB), creatine, creatinine, DNA, fructosamine, glucose, glucose derivatives, glutamine, growth hormones, hormones, ketones, ketone bodies, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In embodiments that monitor more than one analyte, the analytes may be monitored at the same or different times. In one embodiment, the analyte is glucose.

Measuring the concentration of the analyte in the presented interstitial fluid may be performed as described above in the section entitled “Methods of Measuring the Concentration of Analytes in Interstitial Fluid Samples”. It will be understood that the above description of this step also describes various embodiments of the methods of determining accuracy of a subcutaneously implanted analyte sensor. In certain aspects, the concentration of the analyte in the presented interstitial fluid is measured chromatographically, e.g., by high-performance liquid chromatography or ion chromatography. The methods may further include diluting the presented interstitial fluid prior to measuring the concentration of the analyte in the presented interstitial fluid. The reference interstitial fluid analyte concentration, as initially measured, may be normalized, e.g., to control for evaporation and/or variability in sample handling. Also as described above, normalization may include determining the concentration of a secondary (or “reference”) analyte in the presented interstitial fluid. The reference analyte may be a physiological salt component, e.g., a salt component selected from sodium, potassium, magnesium, calcium, chloride, hydrogen phosphate, and hydrogen carbonate.

As noted above, the methods of determining accuracy of a subcutaneously implanted analyte sensor include comparing the interstitial fluid analyte concentration from the subcutaneously implanted analyte sensor to the reference interstitial fluid analyte concentration, thereby determining the accuracy of the subcutaneously implanted analyte sensor. Comparing the concentration from the analyte sensor (the “sensor concentration”) and the reference interstitial fluid analyte concentration (the “reference concentration”) may involve determining the difference between the two concentrations, e.g., the absolute value of the difference. Generally, it will be assumed that when the sensor concentration differs from reference concentration, the reference concentration more accurately reflects the in vivo interstitial fluid analyte concentration of the subject. As such, comparing the sensor and reference concentrations permits determination of the accuracy of the subcutaneously implanted analyte sensor, e.g., as indicated by the magnitude of the difference between the two concentrations.

The level of accuracy of the subcutaneously-implanted analyte sensor may be assessed in a variety of ways. For example, sensor concentrations and reference concentrations may be obtained from multiple subjects, e.g., 2 or more, 5 or more, 10 or more, 20 or more, 50 or more, 100 or more, 250 or more, 500 or more, e.g., a plurality of subjects. For each subject, the sensor concentration may be compared to the reference concentration. In certain embodiments of the present disclosure, 80% of the analyte concentration measurements from the subcutaneously implanted analyte sensor deviate from the reference interstitial fluid analyte concentrations by 25% or less, by 20% or less, by 15% or less, by 10% or less, by 5% or less, by 2.5% or less, by 1% or less, or by 0.5% or less. In other embodiments, 90% of the analyte concentration measurements from the subcutaneously implanted analyte sensor deviate from the reference interstitial fluid analyte concentrations by 25% or less, by 20% or less, by 15% or less, by 10% or less, by 5% or less, by 2.5% or less, by 1% or less, or by 0.5% or less. Determining the accuracy of the sensor readings (e.g., the clinical accuracy), may be carried out by comparing the sensor concentrations and the reference interstitial fluid analyte concentrations using the Clarke Error Grid analysis. See Clarke, et al., “Evaluating Clinical Accuracy of Systems for Self-Monitoring of Blood Glucose” Diabetes Care 10(5): 622-628 (1987).

Accuracy may be assessed in other ways as well. For example, the readings of two or more (e.g., two, three, four, etc.) subcutaneously implanted analyte sensors (e.g., analyte sensors from the same or different manufacturing lots) may be obtained from a single subject within whom the two or more subcutaneous analyte sensors are implanted simultaneously. One or multiple (e.g., a plurality of) concentration measurements from each of the analyte sensors may be combined and compared to one or more reference analyte interstitial fluid concentrations taken from the same subject. In other aspects, the accuracy of each analyte sensor may be assessed individually. When the simultaneously implanted analyte sensors are from the same manufacturing lot, the accuracy assessment may be indicative of the accuracy of that manufacturing lot generally, or the degree of variability between analyte sensors of that lot. Consistency between lots may be assessed as well.

Methods of Determining Calibration Factors for Subcutaneously Implanted Analyte Sensors

As summarized above, methods of determining a calibration factor for a subcutaneously implanted analyte sensor are provided. The methods include determining an interstitial fluid analyte concentration from a subject using the subcutaneously implanted analyte sensor. The methods also include determining a reference interstitial fluid analyte concentration from the subject, where the determining includes abrading an epidermal skin layer of the subject at a first site, applying a vacuum to the abraded epidermal skin layer such that interstitial fluid presents on a surface of the abraded epidermal skin layer, and measuring the concentration of the analyte in the presented interstitial fluid, e.g., by chromatography (e.g., ion chromatography, HPLC, etc.), a test strip and corresponding meter capable of measuring analyte levels in interstitial fluid, quantitative electrophoresis-based assays (e.g., where the target analyte is labeled (e.g., radiolabeled, fluorescently-labeled, etc.), quantitative spectroscopic-based assays, mass spectrometry, quantitative colorimetric assays, noninvasive approaches (e.g., using Raman spectrometers, or any other electromagnetic radiation-based devices configured to non-invasively determine the concentration of an analyte in blood, interstitial fluid, or other bodily fluid or tissue), combinations thereof, etc. The methods further include comparing the interstitial fluid analyte concentration from the subcutaneously implanted analyte sensor to the reference interstitial fluid analyte concentration to determine a difference between the concentrations, and determining the calibration factor for the subcutaneously implanted analyte sensor based on the difference.

Determining an interstitial fluid analyte concentration from a subject using the subcutaneously implanted analyte sensor may be performed as described above in the section entitled “Methods of Determining the Accuracy of Subcutaneously Implanted Analyte Sensors”, and also as described in detail herein below. It will be understood that the above description of this step also describes various embodiments of the methods of calibrating a subcutaneously implanted analyte sensor. Briefly, analyte sensors that find use in the subject methods include, but are not limited to, in vivo analyte sensors that are at least partially implantable within the body of the subject. For example, the analyte sensor may be an electrochemical biosensor adapted for insertion into a subcutaneous site within the body and designed, e.g., for the continuous monitoring of analyte levels in the interstitial fluid of the subcutaneous site. In certain aspects, the sensor operates to electrolyze an analyte of interest in the subcutaneous fluid such that a current is generated between a working electrode and a counter electrode of the sensor. The amount of current generated may correlate to the concentration of the analyte in the interstitial fluid. As such, determining an interstitial fluid analyte concentration from a subject using the subcutaneously implanted analyte sensor may include correlating an electrical signal (e.g., a current) passing through a working electrode to an amount, concentration, or level of an analyte in the interstitial fluid.

Determining a reference interstitial fluid analyte concentration from the subject includes the sub-steps of abrading an epidermal skin layer of the subject at a first site, applying a vacuum to the abraded epidermal skin layer such that interstitial fluid presents on a surface of the abraded epidermal skin layer, and measuring the concentration of the analyte in the presented interstitial fluid. Each of these sub-steps is described in detail above in the section entitled “Methods of Measuring the Concentration of Analytes in Interstitial Fluid Samples”. It will be understood that the above description of these sub-steps also describes various embodiments of the methods of calibrating a subcutaneously implanted analyte sensor.

Embodiments relating to the abrading sub-step are as described above. For example, the abrading may be performed using a rotary device, e.g., a rotary device that includes a rotatable grindstone. The rotary device optionally includes a housing that prevents the rotatable grindstone from abrading a blood vessel, e.g., a capillary beneath the epidermal skin layer. In certain aspects, the abrading includes abrading the epidermal skin layer at a depth ranging from about 0.5 to 3 mm. In other aspects, the abrading includes abrading the stratum corneum. The first site may be located at an area of the subject selected from a forearm, an upper arm, and an abdomen. The abrading may further include abrading an epidermal skin layer of the subject at a second site, e.g., a second site that is adjacent to, or contiguous with, the first site.

Depending on the nature of the analyte, etc., the validity of comparing the concentration measurements from the analyte sensor and the reference interstitial fluid sample may be enhanced by obtaining the reference sample within close proximity to the analyte sensor, e.g., to control for physiological differences between different body regions that may affect the interstitial fluid analyte concentration in a region-specific manner. Accordingly, in one embodiment, the abrading occurs near the location of the subject where the subcutaneous analyte sensor is implanted. For example, if the analyte sensor is subcutaneously implanted in the right forearm of the subject, the abrading may occur on the right forearm of the subject as well (e.g., adjacent to the analyte sensor), and so forth.

Embodiments relating to the sub-step of applying a vacuum are as described above. For example, applying a vacuum pressure optionally includes applying a pressure that does not cause blood to present on the abraded epidermal skin layer. In certain aspects, applying a vacuum to the abraded epidermal skin layer comprises applying a vacuum pressure ranging from about 6 to 20 inches of mercury.

The analyte to be measured may be any analyte of interest, including but not limited to acetyl choline, amylase, bilirubin, cholesterol, chorionic gonadotropin, glycosylated hemoglobin (HbA1c), creatine kinase (e.g., CK-MB), creatine, creatinine, DNA, fructosamine, glucose, glucose derivatives, glutamine, growth hormones, hormones, ketones, ketone bodies, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In embodiments that monitor more than one analyte, the analytes may be monitored at the same or different times. In one embodiment, the analyte is glucose.

Measuring the concentration of the analyte in the presented interstitial fluid may be performed as described above in the section entitled “Methods of Measuring the Concentration of Analytes in Interstitial Fluid Samples”. It will be understood that the above description of this step also describes various embodiments of the methods of calibrating a subcutaneously implanted analyte sensor. In certain aspects, the concentration of the analyte in the presented interstitial fluid is measured chromatographically, e.g., by high-performance liquid chromatography or ion chromatography. The methods may further include diluting the presented interstitial fluid prior to measuring the concentration of the analyte in the presented interstitial fluid. The reference interstitial fluid analyte concentration, as initially measured, may be normalized, e.g., to control for evaporation and/or variability in sample handling. As described above, normalization may include determining the concentration of a secondary (or “reference”) analyte in the presented interstitial fluid. The reference analyte may be a physiological salt component, e.g., a salt component selected from sodium, potassium, magnesium, calcium, chloride, hydrogen phosphate, and hydrogen carbonate.

The step involving comparing the interstitial fluid analyte concentration from the subcutaneously implanted analyte sensor to the reference interstitial fluid analyte concentration may be performed according to the description in the above section entitled “Methods of Determining the Accuracy of Subcutaneously Implanted Analyte Sensors”. It will be understood that the above description of this step also describes various embodiments of the methods of calibrating a subcutaneously implanted analyte sensor. In one embodiment, the difference between the concentrations may be determined by subtracting, e.g., the interstitial fluid analyte concentration from the subcutaneously implanted analyte sensor (the “sensor concentration”) from the reference interstitial fluid analyte concentration (the “reference concentration”). Alternatively, the reference concentration may be subtracted from the sensor concentration. The difference between the two concentrations may be expressed in terms of the absolute value of the difference. As described above, the difference may also be assessed by comparing multiple sensor concentration measurements to multiple corresponding reference concentration measurement. For example, in certain embodiments, 90% of the analyte concentration measurements from the subcutaneously implanted analyte sensor deviate from the reference interstitial fluid analyte concentrations by 25% or less, by 20% or less, by 15% or less, by 10% or less, by 5% or less, by 2.5% or less, by 1% or less, or by 0.5% or less. Determining the accuracy of the sensor readings (e.g., the clinical accuracy), may be carried out by comparing the sensor concentrations and the reference interstitial fluid analyte concentrations using the Clarke Error Grid analysis. See Clarke, et al., “Evaluating Clinical Accuracy of Systems for Self-Monitoring of Blood Glucose” Diabetes Care 10(5): 622-628 (1987).

Determining the calibration factor for the subcutaneously implanted analyte sensor based on the difference between the sensor and reference concentrations may be accomplished in a variety of ways, and may be dependent on the type of analyte sensor and how signals generated by the sensor (e.g., electrical signals in the case of an electrochemical sensor) are correlated to the in vivo interstitial fluid analyte concentration of the subject.

Methods of Determining the Concentration of Analytes in Bodily Samples

Also provided are methods of determining the concentration of an analyte in a bodily sample. The methods include measuring the concentration of an analyte in the sample (e.g., using any of the other analyte concentration-determining methods disclosed herein, such as ion chromatography), measuring the concentration of a physiological salt component in the sample, and normalizing the analyte concentration based on the concentration of the physiological salt component, thereby determining the concentration of the analyte in the bodily sample.

The bodily sample may be any sample for which determining the concentration of an analyte may benefit from normalizing a measured analyte concentration based on the concentration of a physiological salt component. For example, the bodily sample may be a bodily fluid, e.g., blood, interstitial fluid, tears, saliva, urine, plasma, perspiration, cerebrospinal fluid, a cell lysate, and the like.

According to one embodiment, the bodily sample is an interstitial fluid sample. When the bodily sample is an interstitial fluid sample, the methods optionally include obtaining the interstitial fluid sample by abrading an epidermal skin layer of a subject, applying a vacuum to the abraded epidermal skin layer such that the interstitial fluid sample presents on a surface of the abraded epidermal skin layer, and collecting the interstitial fluid sample presented on the surface. The abrading, applying a vacuum, and collecting steps may be performed as described above in the section entitled “Methods of Measuring the Concentration of Analytes in Interstitial Fluid Samples”. It will be understood that the above description of these steps also describes certain embodiments of the methods of determining the concentration of an analyte in a bodily sample of the present section.

The analyte of which the concentration is to be determined may be any analyte of interest, including but not limited to acetyl choline, amylase, bilirubin, cholesterol, chorionic gonadotropin, glycosylated hemoglobin (HbA1c), creatine kinase (e.g., CK-MB), creatine, creatinine, DNA, fructosamine, glucose, glucose derivatives, glutamine, growth hormones, hormones, ketones, ketone bodies, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In embodiments that monitor more than one analyte, the analytes may be monitored at the same or different times. In one embodiment, the analyte is glucose.

Measuring the concentration of the analyte, e.g., in an interstitial fluid sample, may be performed as described above in the section entitled “Methods of Measuring the Concentration of Analytes in Interstitial Fluid Samples”. It will be understood that the above description of this step also describes various embodiments of the methods of determining the concentration of an analyte in a bodily sample.

As noted above, the concentrations of physiological salts are substantially constant in certain bodily fluids, e.g., interstitial fluid. In one embodiment, the physiological salt component is optionally selected from sodium, potassium, magnesium, calcium, chloride, hydrogen phosphate, and hydrogen carbonate. By way of example, the concentration of the analyte of interest (e.g., glucose) may be measured in the interstitial fluid sample, e.g., by ion chromatography, and the sodium concentration in the same sample can be determined, e.g., by ion chromatography or any other convenient and reliable means. Any disparity between the known (“constant”) concentration of sodium in interstitial fluid and the measured sodium concentration of the interstitial fluid sample may be used to normalize the concentration measurement for glucose. For example, if the measured sodium concentration is greater than the known concentration of sodium in interstitial fluid by a factor of 2, the measured glucose concentration can be divided by 2 to obtain a normalized glucose concentration that accurately indicates the glucose concentration in the interstitial fluid of the subject.

Methods of Extracting Interstitial Fluid

As summarized above, methods of extracting interstitial fluid are provided. In one embodiment, the methods include abrading an epidermal skin layer of a subject at a first site, and applying a vacuum to the abraded epidermal skin layer such that interstitial fluid presents on a surface of the abraded epidermal skin layer.

The steps of abrading and applying a vacuum may be performed as described above in the section entitled “Methods of Measuring the Concentration of Analytes in Interstitial Fluid Samples”. It will be understood that the above description of these steps also describes various embodiments of the methods of extracting interstitial fluid of the present section.

Embodiments relating to the abrading step are as described above. For example, the abrading may be performed using a rotary device, e.g., a rotary device that includes a rotatable grindstone. The rotary device optionally includes a housing that prevents the rotatable grindstone from abrading a blood vessel, e.g., a capillary beneath the epidermal skin layer. In certain aspects, the abrading includes abrading the epidermal skin layer at a depth ranging from about 0.5 to 3 mm. In other aspects, the abrading includes abrading the stratum corneum. The first site may be located at an area of the subject selected from a forearm, an upper arm, and an abdomen. The abrading may further include abrading an epidermal skin layer of the subject at a second site, e.g., a second site that is adjacent to, or contiguous with, the first site.

Embodiments relating to the step of applying a vacuum are as described above. For example, applying a vacuum optionally includes applying a vacuum pressure that does not cause blood to present on the abraded epidermal skin layer. In certain aspects, applying a vacuum to the abraded epidermal skin layer comprises applying a vacuum pressure ranging from about 6 to 20 inches of mercury.

Methods of Manufacturing

Also provided by the present disclosure are methods of manufacturing. In certain aspects, the methods involve assembling a first in vivo analyte sensor, determining the accuracy of the sensor, and assembling one or more additional in vivo analyte sensors based on the accuracy of the first sensor. The formation and/or assembly of the one or more additional analyte sensors may be informed by the accuracy determination of the first analyte sensor. For example, if it is determined that the first analyte sensor provides an analyte concentration that is above or below the actual analyte concentration (e.g., as determined by comparing the concentration to a concentration determined from a reference sample by, e.g., ion chromatography), the method of forming and/or assembling of the one or more additional analyte sensors may be adjusted to correct for the high or low sensor reading. In the case of a reading that is too low, a number of manufacturing adjustments may be made, e.g., increasing the amount of sensing reagent (e.g., analyte-responsive enzyme, redox mediator, or both) on the sensor(s) (e.g., by disposing more sensing reagent on a working electrode of the sensor, disposing a sensing reagent of higher concentration on a working electrode of the sensor, etc.), decreasing the thickness of an analyte flux limiting membrane disposed over a sensing region of the sensor(s), increasing an area of a working electrode on the sensor(s), etc. Similarly, manufacturing adjustments may be made when the first analyte sensor provides a reading that is too high. For example, making the one or more additional sensors may involve, e.g., decreasing the amount of sensing reagent (e.g., analyte-responsive enzyme, redox mediator, or both) on the sensor(s) (e.g., by disposing less sensing reagent on a working electrode of the sensor, disposing a sensing reagent of lesser concentration on a working electrode of the sensor, etc.), increasing the thickness of an analyte flux limiting membrane disposed over a sensing region of the sensor(s), decreasing an area of a working electrode on the sensor(s), etc. Alternatively, or additionally, the one or more additional analyte sensors may be assembled and operably coupled to a sensor control unit having a memory in which a calibration factor is stored, the calibration factor correcting for the high or low reading obtained using the first analyte sensor.

The in vivo analyte sensors made according to the methods of manufacturing disclosed herein may be configured to determine the concentration of any analyte of interest, including but not limited to acetyl choline, amylase, bilirubin, cholesterol, chorionic gonadotropin, glycosylated hemoglobin (HbA1c), creatine kinase (e.g., CK-MB), creatine, creatinine, DNA, fructosamine, glucose, glucose derivatives, glutamine, growth hormones, hormones, ketones, ketone bodies, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In embodiments that monitor more than one analyte, the analytes may be monitored at the same or different times.

According to a first embodiment, provided is a method of manufacturing that includes assembling a first in vivo analyte sensor including a sensor control unit and an subcutaneously implantable analyte sensor, and determining accuracy of the first in vivo analyte sensor. Determining the accuracy of the first in vivo analyte sensor includes subcutaneously implanting the first analyte sensor in a subject, determining an interstitial fluid analyte concentration from the subject using the first analyte sensor, determining a reference interstitial fluid analyte concentration from the subject, and comparing the interstitial fluid analyte concentration from the first analyte sensor to the reference interstitial fluid analyte concentration, thereby determining the accuracy of the subcutaneously implanted analyte sensor. Optionally, the method further includes deriving a calibration factor based on a difference between the interstitial fluid analyte concentration from the first analyte sensor and the reference interstitial fluid analyte concentration. Methods that include deriving a calibration factor may further include assembling a second in vivo analyte sensor that includes a sensor control unit, the sensor control unit including a memory that includes the calibration factor.

When practicing the methods of this first embodiment, the first analyte sensor may include a first amount of sensing reagent including an analyte-responsive enzyme and a redox mediator, where the method further includes assembling a second in vivo analyte sensor that includes a second amount of sensing reagent, the second amount of sensing reagent being different from the first amount of sensing reagent based on a difference between the interstitial fluid analyte concentration from the first analyte sensor and the reference interstitial fluid analyte concentration.

Adjusting the amount of sensing reagent included on the second in vivo analyte sensor may be effected by a variety of approaches. For example, the concentration of a sensing reagent (e.g., the concentration of an analyte-responsive enzyme and/or redox mediator) in a solution applied to a working electrode of the second analyte sensor may be increased or decreased when the first analyte sensor generates a reading that is too low or too high, respectively, as determined by comparing the reading of the first analyte sensor to the reference interstitial fluid analyte concentration. Alternatively, or additionally, the sensing reagent may be applied to a greater area or lesser area of a working electrode of the second analyte sensor when the first analyte sensor generates a reading that is too low or too high, respectively, as determined by comparing the reading of the first analyte sensor to the reference interstitial fluid analyte concentration. In certain aspects, the sensing reagent is disposed as one or more discrete spots on the surface of a working electrode of the first in vivo analyte sensor. During manufacture of the second in vivo analyte sensor, the number and/or size of spots of sensing reagent disposed on a working electrode of the second sensor may be increased or decreased when the first analyte sensor generates a reading that is too low or too high, respectively, as determined by comparing the reading of the first analyte sensor to the reference interstitial fluid analyte concentration. Analyte sensors that include sensing surfaces (e.g., working electrodes) having discrete spots of sensing reagent are described in U.S. application Ser. No. 13/315,034, the disclosure of which is incorporated herein by reference in its entirety for all purposes.

The first analyte sensor may include a sensing region and a membrane disposed over the sensing region, where a thickness of the membrane determines a rate of flux of the analyte across the membrane. According to this aspect, the method may further include assembling a second in vivo analyte sensor including a sensing region and a membrane having a thickness disposed over the sensing region, where the thickness of the membrane of the second analyte sensor is different from the thickness of the membrane of the first analyte sensor, the difference in thickness being based on a difference between the interstitial fluid analyte concentration from the first analyte sensor and the reference interstitial fluid analyte concentration.

A number of strategies may be employed to increase or decrease the thickness of the membrane disposed over the sensing region of the second in vivo analyte sensor. For example, disposing the membrane over the sensing region may include applying a solution (e.g., a polymer solution such as a crosslinker-polymer solution) to the sensing layer of a subcutaneously implantable analyte sensor, e.g., by placing a droplet or droplets of the solution on the sensor, by dipping the sensor into the solution, or the like. The thickness of the membrane may be controlled, e.g., by the concentration of the solution, by the number of droplets of the solution applied, by the number of times the sensor is dipped in the solution, or by any combination of these factors. In certain aspects, where the first in vivo analyte sensor provides an analyte concentration reading that is too high as determined by comparison to the reference interstitial fluid analyte concentration, the concentration of the solution (e.g., the concentration of a polymer and/or cross-linking component in the solution) applied to the sensing region of the second analyte sensor may be increased, a number of droplets of the solution applied to the sensing region of the second analyte sensor may be increased, and/or the number of times the second analyte sensor is dipped in the solution may be increased. Conversely, where the first in vivo analyte sensor provides an analyte concentration reading that is too low as determined by comparison to the reference interstitial fluid analyte concentration, the concentration of the solution (e.g., the concentration of a polymer and/or cross-linking component in the solution) applied to the sensing region of the second analyte sensor may be reduced, a number of droplets of the solution applied to the sensing region of the second analyte sensor may be reduced, and/or the number of times the second analyte sensor is dipped in the solution may be reduced. Methods for disposing analyte-diffusion-limiting membranes on sensing regions of in vivo analyte sensors are described in U.S. Pat. No. 6,932,894 to Mao et al., the disclosure of which is incorporated herein by reference in its entirety for all purposes.

The first analyte sensor may include a working electrode having an area that determines a signal intensity of the first analyte sensor in response to the analyte. Here, the methods may further include assembling a second in vivo analyte sensor including a working electrode having an area different from the area of the first analyte sensor, the difference in the working electrode areas being based on a difference between the interstitial fluid analyte concentration from the first analyte sensor and the reference interstitial fluid analyte concentration.

When practicing any of the methods of manufacturing described herein, the reference interstitial fluid analyte concentration may be determined using any suitable approaches for obtaining an interstitial fluid sample and measuring the concentration of an analyte therein. For example, determining a reference interstitial fluid analyte concentration from the subject may include abrading an epidermal skin layer of the subject at a first site, applying a vacuum to the abraded epidermal skin layer such that interstitial fluid presents on a surface of the abraded epidermal skin layer, and measuring the concentration of the analyte in the presented interstitial fluid. Each of these sub-steps is described in detail above in the section entitled “Methods of Measuring the Concentration of Analytes in Interstitial Fluid Samples.” It will be understood that the above description of these sub-steps also describes certain aspects of the methods of manufacturing disclosed herein.

According to any of the methods of manufacturing disclosed herein, the concentration of the analyte in the presented interstitial fluid may be measured using any of a variety of approaches, e.g., chromatography (e.g., ion chromatography, HPLC), a test strip and corresponding meter capable of measuring analyte levels in interstitial fluid, quantitative electrophoresis-based assays (e.g., where the target analyte is labeled (e.g., radiolabeled, fluorescently-labeled, etc.)), quantitative spectroscopic-based assays, mass spectrometry, quantitative colorimetric assays, noninvasive approaches (e.g., using Raman spectrometers, or any other electromagnetic radiation-based devices configured to non-invasively determine the concentration of an analyte in blood, interstitial fluid, or other bodily fluid or tissue), combinations thereof, etc. For example, measuring the concentration of the analyte in the presented interstitial fluid may include measuring the concentration by ion chromatography.

The subject methods may further include normalizing the reference interstitial fluid analyte concentration to the concentration of a reference analyte in the reference interstitial fluid sample to control for, e.g., potential evaporation of water (and corresponding concentration of the analyte) during ISF sample acquisition, and any variability in sample handling, etc. Reference analytes for normalizing the reference interstitial fluid analyte concentration may include, but are not limited to, physiological salts such as sodium, potassium, magnesium, calcium, chloride, hydrogen phosphate, hydrogen carbonate, and combinations thereof.

Also provided are methods of manufacturing where determining the accuracy of a first in vivo analyte sensor does not involve subcutaneously implanting the first sensor in a subject. For example, provided is a method of manufacturing that includes assembling a first in vivo analyte sensor comprising a sensor control unit and an subcutaneously implantable analyte sensor, and determining accuracy of the first analyte sensor. According to this embodiment, determining the accuracy of the first analyte sensor includes obtaining an interstitial fluid sample, determining the analyte concentration from a first aliquot of the interstitial fluid sample using the first in vivo analyte sensor (e.g., by applying an amount of the first aliquot of the sample to a sensing region of the sensor, dipping a sensing region of the sensor into the first aliquot of the sample, or the like), determining the analyte concentration from a second aliquot of the interstitial fluid sample using an analyte concentration determining process different than the first analyte sensor, and comparing the interstitial fluid sample analyte concentration from the first analyte sensor and the interstitial fluid sample analyte concentration from the analyte concentration determining process different than the first analyte sensor, thereby determining the accuracy of the first analyte sensor.

Optionally, the method further includes deriving a calibration factor based on a difference between the interstitial fluid sample analyte concentration from the first analyte sensor and the interstitial fluid sample analyte concentration from the analyte concentration determining process different than the first analyte sensor. When the method includes deriving such a calibration factor, the method may further include assembling a second in vivo analyte sensor comprising a sensor control unit, wherein the sensor control unit comprises a memory comprising the calibration factor.

The first analyte sensor may include a first amount of sensing reagent that includes an analyte-responsive enzyme and a redox mediator, where the method further includes assembling a second in vivo analyte sensor including a second amount of sensing reagent, the second amount of sensing reagent being different from the first amount of sensing reagent based on a difference between the interstitial fluid sample analyte concentration from the first analyte sensor and the interstitial fluid sample analyte concentration from the analyte concentration determining process different than the first analyte sensor. Adjusting the amount of sensing reagent included on the second in vivo analyte sensor is described elsewhere herein, as well as in U.S. application Ser. No. 13/315,034, the disclosure of which is incorporated herein by reference in its entirety for all purposes.

The first analyte sensor may include a sensing region and a membrane disposed over the sensing region, where a thickness of the membrane determines a rate of flux of the analyte across the membrane. According to this aspect, the method may further include assembling a second in vivo analyte sensor including a sensing region and a membrane having a thickness disposed over the sensing region, where the thickness of the membrane of the second analyte sensor is different from the thickness of the membrane of the first analyte sensor, the difference in thickness being based on a difference between the interstitial fluid analyte concentration from the first analyte sensor and the interstitial fluid sample analyte concentration from the analyte concentration determining process different than the first analyte sensor. Methods for disposing analyte-diffusion-limiting membranes on sensing regions of in vivo analyte sensors are described elsewhere herein, as well as in U.S. Pat. No. 6,932,894 to Mao et al., the disclosure of which is incorporated herein by reference in its entirety for all purposes.

The first analyte sensor may include a working electrode having an area that determines a signal intensity of the first analyte sensor in response to the analyte. Here, the methods may further include assembling a second in vivo analyte sensor including a working electrode having an area different from the area of the first analyte sensor, the difference in the working electrode areas being based on a difference between the interstitial fluid sample analyte concentration from the first analyte sensor and the interstitial fluid sample analyte concentration from the analyte concentration determining process different than the first analyte sensor.

Obtaining the interstitial fluid sample may include, e.g., abrading an epidermal skin layer of the subject at a first site, applying a vacuum to the abraded epidermal skin layer such that interstitial fluid presents on a surface of the abraded epidermal skin layer, and collecting the presented interstitial fluid. Each of these sub-steps is described in detail above in the section entitled “Methods of Measuring the Concentration of Analytes in Interstitial Fluid Samples.” It will be understood that the above description of these sub-steps also describes certain aspects of the methods of manufacturing disclosed herein.

The analyte concentration determining process different than the first analyte sensor may include, but is not limited to, e.g., chromatography (e.g., ion chromatography, HPLC), quantitative electrophoresis-based assays (e.g., where the target analyte is labeled (e.g., radiolabeled, fluorescently-labeled, etc.), quantitative spectroscopic-based assays, mass spectrometry, quantitative colorimetric assays, noninvasive approaches (e.g., using Raman spectrometers, or any other electromagnetic radiation-based devices configured to non-invasively determine the concentration of an analyte in blood, interstitial fluid, or other bodily fluid or tissue), combinations thereof, etc. For example, measuring the concentration of the analyte in the presented interstitial fluid may include measuring the concentration by ion chromatography.

The subject methods may further include normalizing the interstitial fluid analyte concentration to the concentration of a reference analyte in the reference interstitial fluid sample to control for, e.g., potential evaporation of water (and corresponding concentration of the analyte) during ISF sample acquisition, and any variability in sample handling, etc. Reference analytes for normalizing the reference interstitial fluid analyte concentration may include, but are not limited to, physiological salts such as sodium, potassium, magnesium, calcium, chloride, hydrogen phosphate, hydrogen carbonate, and combinations thereof.

Determining the analyte concentration from the second aliquot of the interstitial fluid sample may include normalizing the analyte concentration to the concentration of a reference analyte, including but not limited to, a physiological salt component in the interstitial fluid sample. Such physiological salts include, e.g., sodium, potassium, magnesium, calcium, chloride, hydrogen phosphate, hydrogen carbonate, and combinations thereof.

The present disclosure also provides sensors made according to the methods of manufacturing disclosed herein. According to certain embodiments, the sensor is a subcutaneously implantable analyte sensor, where the sensor has an accuracy that is at least 70% (e.g., at least 70%, at least 80%, at least 90%, at least 95%, e.g., at least 99%) as determined by comparing an interstitial fluid analyte concentration reading obtained using the sensor to an interstitial fluid analyte concentration reading obtained using a different analyte concentration determining process (e.g., an analyte concentration determining process other than the analyte sensor). The interstitial fluid analyte concentration reading obtained using the sensor may be obtained by subcutaneously implanting the analyte sensor in a subject (e.g., a human subject). The different analyte concentration determining process may include, e.g., chromatography (e.g., ion chromatography, HPLC), a test strip and corresponding meter capable of measuring analyte levels in interstitial fluid, quantitative electrophoresis-based assays (e.g., where the target analyte is labeled (e.g., radiolabeled, fluorescently-labeled, etc.), quantitative spectroscopic-based assays, mass spectrometry, quantitative colorimetric assays, noninvasive approaches (e.g., using Raman spectrometers, or any other electromagnetic radiation-based devices configured to non-invasively determine the concentration of an analyte in blood, interstitial fluid, or other bodily fluid or tissue), combinations thereof, etc. For example, measuring the concentration of the analyte in the presented interstitial fluid may include measuring the concentration by ion chromatography. In one aspect, the different analyte concentration determining process is an ion chromatography system.

Also provided are in vivo analyte sensors including a sensor control unit and a subcutaneously implantable analyte sensor, where the sensor control unit includes a memory including a calibration factor based on a difference between an interstitial fluid sample analyte concentration from a first in vivo analyte sensor and an interstitial fluid sample analyte concentration from an interstitial fluid analyte concentration reading obtained using a different analyte concentration determining process. The interstitial fluid sample analyte concentration reading obtained using a different analyte concentration determining process is determined from an interstitial fluid sample obtained by abrading an epidermal skin layer of a subject at a first site, applying a vacuum to the abraded epidermal skin layer such that interstitial fluid presents on a surface of the abraded epidermal skin layer, and collecting the presented interstitial fluid. The different analyte concentration determining process may be ion chromatography, or any other convenient analyte concentration determining process, e.g., those described elsewhere herein. In certain aspects, the different analyte concentration determining process includes normalizing the analyte concentration to the concentration of a reference analyte, e.g., one or more physiological salt component in the interstitial fluid sample. The physiological salt component(s) may include, but is not limited to, sodium, potassium, magnesium, calcium, chloride, hydrogen phosphate, hydrogen carbonate, and combinations thereof.

### Utility

The subject methods find use in a variety of different applications where, e.g., the accurate determination of an analyte concentration by an analyte sensor is desired. For example, the methods are useful for obtaining and accurately determining the concentration of one or more analytes in a reference bodily sample, e.g., a reference interstitial fluid sample. The measured concentration of one or more analytes in the reference bodily sample can be used, e.g., to determine the accuracy of (and if desired, calibrate) an analyte sensor or other component to be used, e.g., by a patient as part of an in vivo analyte monitoring system (e.g., a continuous and/or automatic glucose monitoring system).

### Analyte Sensors and Monitoring Systems

Embodiments of the present disclosure relate to methods for detecting at least one analyte, including glucose, in body fluid. Embodiments relate to the continuous and/or automatic in vivo monitoring of the level of one or more analytes using a continuous analyte monitoring system that includes an analyte sensor at least a portion of which is to be positioned beneath a skin surface of a user for a period of time. Embodiments include combined or combinable devices, systems and methods and/or transferring data between an in vivo continuous system and an in vivo system. In some embodiments, the systems, or at least a portion of the systems, are integrated into a single unit.

A sensor as described herein may be an in vivo sensor or an in vitro sensor (i.e., a discrete monitoring test strip). Such a sensor can be formed on a substrate, e.g., a substantially planar substrate. In certain embodiments, the sensor is a wire, e.g., a working electrode wire inner portion with one or more other electrodes associated (e.g., on, including wrapped around) therewith. The sensor may also include at least one counter electrode (or counter/reference electrode) and/or at least one reference electrode or at least one reference/counter electrode.

Accordingly, embodiments include analyte monitoring devices and systems that include an analyte sensor at least a portion of which is positionable beneath the skin surface of the user for the in vivo detection of an analyte, including glucose, lactate, and the like, in a body fluid. Embodiments include wholly implantable analyte sensors and analyte sensors in which only a portion of the sensor is positioned under the skin and a portion of the sensor resides above the skin, e.g., for contact to a sensor control unit (which may include a transmitter), a receiver/display unit, transceiver, processor, etc. The sensor may be, for example, subcutaneously positionable in a user for the continuous or periodic monitoring of a level of an analyte in the user's interstitial fluid. For the purposes of this description, continuous monitoring and periodic monitoring will be used interchangeably, unless noted otherwise. The sensor response may be correlated and/or converted to analyte levels in blood or other fluids. In certain embodiments, an analyte sensor may be positioned in contact with interstitial fluid to detect the level of glucose, which detected glucose may be used to infer the glucose level in the user's bloodstream. Analyte sensors may be insertable into a vein, artery, or other portion of the body containing fluid. Embodiments of the analyte sensors may be configured for monitoring the level of the analyte over a time period which may range from seconds, minutes, hours, days, weeks, to months, or longer.

In certain embodiments, the analyte sensors, such as glucose sensors, are capable of in vivo detection of an analyte for one hour or more, e.g., a few hours or more, e.g., a few days or more, e.g., three or more days, e.g., five days or more, e.g., seven days or more, e.g., several weeks or more, or one month or more. Future analyte levels may be predicted based on information obtained, e.g., the current analyte level at time t0, the rate of change of the analyte, etc. Predictive alarms may notify the user of a predicted analyte level that may be of concern in advance of the user's analyte level reaching the future predicted analyte level. This provides the user an opportunity to take corrective action.

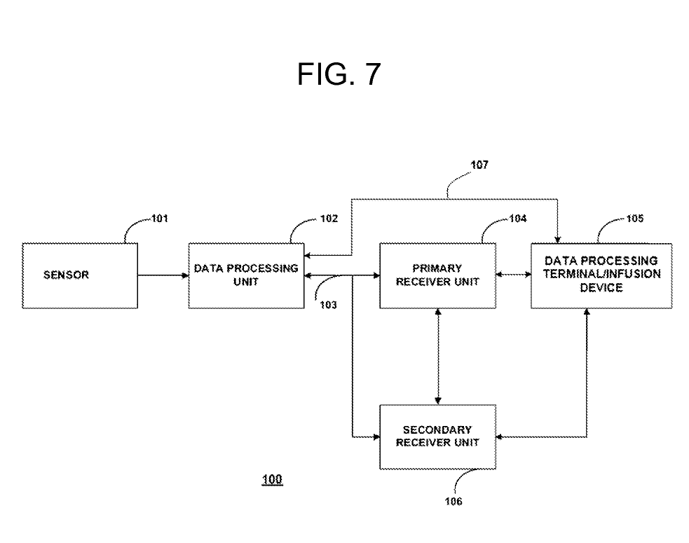

FIG. 7 shows a data monitoring and management system such as, for example, an analyte (e.g., glucose) monitoring system 100 in accordance with certain embodiments. Aspects of the subject disclosure are further described primarily with respect to glucose monitoring devices and systems, and methods of glucose detection, for convenience only and such description is in no way intended to limit the scope of the embodiments. It is to be understood that the analyte monitoring system may be configured to monitor a variety of analytes at the same time or at different times.

Analytes that may be monitored include, but are not limited to acetyl choline, amylase, bilirubin, cholesterol, chorionic gonadotropin, glycosylated hemoglobin (HbA1c), creatine kinase (e.g., CK-MB), creatine, creatinine, DNA, fructosamine, glucose, glucose derivatives, glutamine, growth hormones, hormones, ketones, ketone bodies, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In embodiments that monitor more than one analyte, the analytes may be monitored at the same or different times.

The analyte monitoring system 100 includes an analyte sensor 101, a data processing unit 102 connectable to the sensor 101, and a primary receiver unit 104. In some instances, the primary receiver unit 104 is configured to communicate with the data processing unit 102 via a communication link 103. In certain embodiments, the primary receiver unit 104 may be further configured to transmit data to a data processing terminal 105 to evaluate or otherwise process or format data received by the primary receiver unit 104. The data processing terminal 105 may be configured to receive data directly from the data processing unit 102 via a communication link 107, which may optionally be configured for bi-directional communication. Further, the data processing unit 102 may include a transmitter or a transceiver to transmit and/or receive data to and/or from the primary receiver unit 104 and/or the data processing terminal 105 and/or optionally a secondary receiver unit 106.

Also shown in FIG. 7 is an optional secondary receiver unit 106 which is operatively coupled to the communication link 103 and configured to receive data transmitted from the data processing unit 102. The secondary receiver unit 106 may be configured to communicate with the primary receiver unit 104, as well as the data processing terminal 105. In certain embodiments, the secondary receiver unit 106 may be configured for bi-directional wireless communication with each of the primary receiver unit 104 and the data processing terminal 105. As discussed in further detail below, in some instances, the secondary receiver unit 106 may be a de-featured receiver as compared to the primary receiver unit 104, for instance, the secondary receiver unit 106 may include a limited or minimal number of functions and features as compared with the primary receiver unit 104. As such, the secondary receiver unit 106 may include a smaller (in one or more, including all, dimensions), compact housing or embodied in a device including a wrist watch, arm band, PDA, mp3 player, cell phone, etc., for example. Alternatively, the secondary receiver unit 106 may be configured with the same or substantially similar functions and features as the primary receiver unit 104. The secondary receiver unit 106 may include a docking portion configured to mate with a docking cradle unit for placement by, e.g., the bedside for night time monitoring, and/or a bi-directional communication device. A docking cradle may recharge a power supply.

Only one analyte sensor 101, data processing unit 102 and data processing terminal 105 are shown in the embodiment of the analyte monitoring system 100 illustrated in FIG. 7. However, it will be appreciated by one of ordinary skill in the art that the analyte monitoring system 100 may include more than one sensor 101 and/or more than one data processing unit 102, and/or more than one data processing terminal 105. Multiple sensors may be positioned in a user for analyte monitoring at the same or different times. In certain embodiments, analyte information obtained by a first sensor positioned in a user may be employed as a comparison to analyte information obtained by a second sensor. This may be useful to confirm or validate analyte information obtained from one or both of the sensors. Such redundancy may be useful if analyte information is contemplated in critical therapy-related decisions. In certain embodiments, a first sensor may be used to calibrate a second sensor.

The analyte monitoring system 100 may be a continuous monitoring system, or semi-continuous, or a discrete monitoring system. In a multi-component environment, each component may be configured to be uniquely identified by one or more of the other components in the system so that communication conflict may be readily resolved between the various components within the analyte monitoring system 100. For example, unique IDs, communication channels, and the like, may be used.

In certain embodiments, the sensor 101 is physically positioned in or on the body of a user whose analyte level is being monitored. The sensor 101 may be configured to at least periodically sample the analyte level of the user and convert the sampled analyte level into a corresponding signal for transmission by the data processing unit 102. The data processing unit 102 is coupleable to the sensor 101 so that both devices are positioned in or on the user's body, with at least a portion of the analyte sensor 101 positioned transcutaneously. The data processing unit may include a fixation element, such as an adhesive or the like, to secure it to the user's body. A mount (not shown) attachable to the user and mateable with the data processing unit 102 may be used. For example, a mount may include an adhesive surface. The data processing unit 102 performs data processing functions, where such functions may include, but are not limited to, filtering and encoding of data signals, each of which corresponds to a sampled analyte level of the user, for transmission to the primary receiver unit 104 via the communication link 103. In some embodiments, the sensor 101 or the data processing unit 102 or a combined sensor/data processing unit may be wholly implantable under the skin surface of the user.

In certain embodiments, the primary receiver unit 104 may include an analog interface section including an RF receiver and an antenna that is configured to communicate with the data processing unit 102 via the communication link 103, and a data processing section for processing the received data from the data processing unit 102 including data decoding, error detection and correction, data clock generation, data bit recovery, etc., or any combination thereof.

In operation, the primary receiver unit 104 in certain embodiments is configured to synchronize with the data processing unit 102 to uniquely identify the data processing unit 102, based on, for example, an identification information of the data processing unit 102, and thereafter, to periodically receive signals transmitted from the data processing unit 102 associated with the monitored analyte levels detected by the sensor 101.

Referring again to FIG. 7, the data processing terminal 105 may include a personal computer, a portable computer including a laptop or a handheld device (e.g., a personal digital assistant (PDA), a telephone including a cellular phone (e.g., a multimedia and Internet-enabled mobile phone including an iPhone™, a Blackberry®, or similar phone), an mp3 player (e.g., an iPOD™, etc.), a pager, and the like), and/or a drug delivery device (e.g., an infusion device), each of which may be configured for data communication with the receiver via a wired or a wireless connection. Additionally, the data processing terminal 105 may further be connected to a data network (not shown) for storing, retrieving, updating, and/or analyzing data corresponding to the detected analyte level of the user.

The data processing terminal 105 may include a drug delivery device (e.g., an infusion device), such as an insulin infusion pump or the like, which may be configured to administer a drug (e.g., insulin) to the user, and which may be configured to communicate with the primary receiver unit 104 for receiving, among others, the measured analyte level. Alternatively, the primary receiver unit 104 may be configured to integrate an infusion device therein so that the primary receiver unit 104 is configured to administer an appropriate drug (e.g., insulin) to users, for example, for administering and modifying basal profiles, as well as for determining appropriate boluses for administration based on, among others, the detected analyte levels received from the data processing unit 102. An infusion device may be an external device or an internal device, such as a device wholly implantable in a user.

In certain embodiments, the data processing terminal 105, which may include an infusion device, e.g., an insulin pump, may be configured to receive the analyte signals from the data processing unit 102, and thus, incorporate the functions of the primary receiver unit 104 including data processing for managing the user's insulin therapy and analyte monitoring. In certain embodiments, the communication link 103, as well as one or more of the other communication interfaces shown in FIG. 7, may use one or more wireless communication protocols, such as, but not limited to: an RF communication protocol, an infrared communication protocol, a Bluetooth enabled communication protocol, an 802.11x wireless communication protocol, or an equivalent wireless communication protocol which would allow secure, wireless communication of several units (for example, per Health Insurance Portability and Accountability Act (HIPPA) requirements), while avoiding potential data collision and interference.

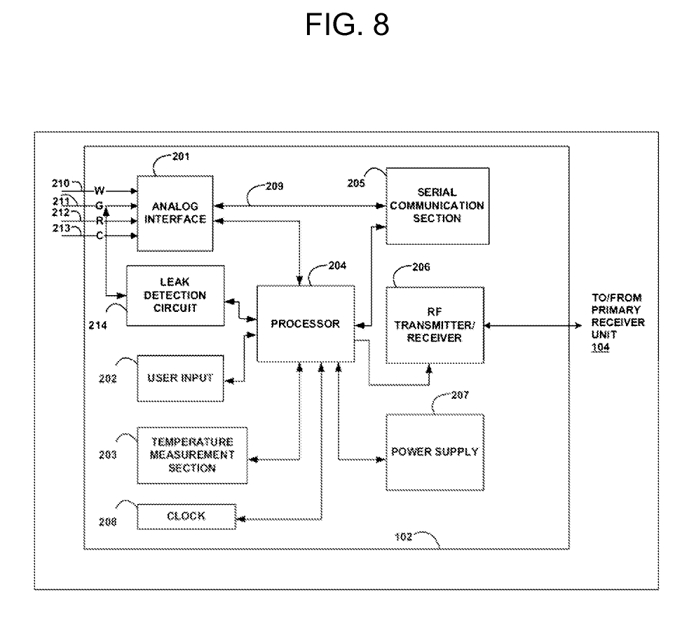

FIG. 8 shows a block diagram of an embodiment of a data processing unit 102 of the analyte monitoring system shown in FIG. 7. User input and/or interface components may be included or a data processing unit may be free of user input and/or interface components. In certain embodiments, one or more application-specific integrated circuits (ASIC) may be used to implement one or more functions or routines associated with the operations of the data processing unit (and/or receiver unit) using for example one or more state machines and buffers.

As can be seen in the embodiment of FIG. 8, the analyte sensor 101 (FIG. 7) includes four contacts, three of which are electrodes: a work electrode (W) 210, a reference electrode (R) 212, and a counter electrode (C) 213, each operatively coupled to the analog interface 201 of the data processing unit 102. This embodiment also shows an optional guard contact (G) 211. Fewer or greater electrodes may be employed. For example, the counter and reference electrode functions may be served by a single counter/reference electrode. In some cases, there may be more than one working electrode and/or reference electrode and/or counter electrode, etc.

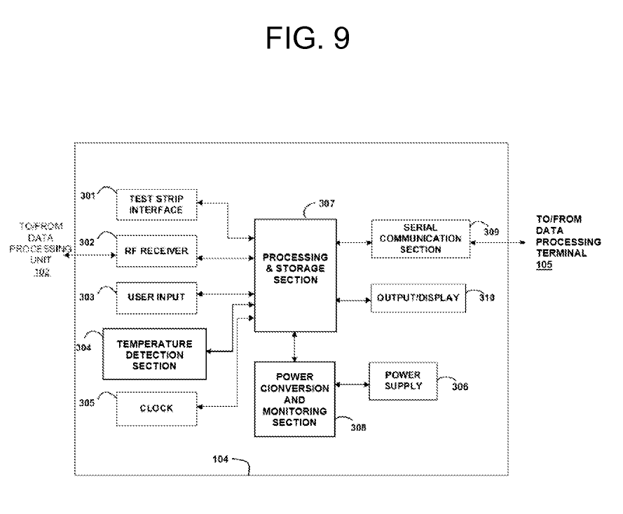

FIG. 9 is a block diagram of an embodiment of a receiver/monitor unit such as the primary receiver unit 104 of the analyte monitoring system shown in FIG. 7. The primary receiver unit 104 includes one or more of: a test strip interface 301, an RF receiver 302, a user input 303, an optional temperature detection section 304, and a clock 305, each of which is operatively coupled to a processing and storage section 307. The primary receiver unit 104 also includes a power supply 306 operatively coupled to a power conversion and monitoring section 308. Further, the power conversion and monitoring section 308 is also coupled to the processing and storage section 307. Moreover, also shown are a receiver serial communication section 309, and an output 310, each operatively coupled to the processing and storage section 307. The primary receiver unit 104 may include user input and/or interface components or may be free of user input and/or interface components.

In certain embodiments, the test strip interface 301 includes an analyte testing portion (e.g., a glucose level testing portion) to receive a blood (or other body fluid sample) analyte test or information related thereto. For example, the test strip interface 301 may include a test strip port to receive a test strip (e.g., a glucose test strip). The device may determine the analyte level of the test strip, and optionally display (or otherwise notice) the analyte level on the output 310 of the primary receiver unit 104. Any suitable test strip may be employed, e.g., test strips that only require a very small amount (e.g., 3 microliters or less, e.g., 1 microliter or less, e.g., 0.5 microliters or less, e.g., 0.1 microliters or less), of applied sample to the strip in order to obtain accurate glucose information. Embodiments of test strips include, e.g., FreeStyle® blood glucose test strips from Abbott Diabetes Care, Inc. (Alameda, Calif.). Glucose information obtained by an in vitro glucose testing device may be used for a variety of purposes, computations, etc. For example, the information may be used to calibrate sensor 101, confirm results of sensor 101 to increase the confidence thereof (e.g., in instances in which information obtained by sensor 101 is employed in therapy related decisions), etc.

In further embodiments, the data processing unit 102 and/or the primary receiver unit 104 and/or the secondary receiver unit 106, and/or the data processing terminal/infusion device 105 may be configured to receive the analyte value wirelessly over a communication link from, for example, a blood glucose meter. In further embodiments, a user manipulating or using the analyte monitoring system 100 (FIG. 7) may manually input the analyte value using, for example, a user interface (for example, a keyboard, keypad, voice commands, and the like) incorporated in one or more of the data processing unit 102, the primary receiver unit 104, secondary receiver unit 106, or the data processing terminal/infusion device 105.

Additional detailed descriptions are provided in U.S. Pat. Nos. 5,262,035; 5,264,104; 5,262,305; 5,320,715; 5,593,852; 6,175,752; 6,650,471; 6,746,582, and 7,811,231, each of which is incorporated herein by reference in their entirety.

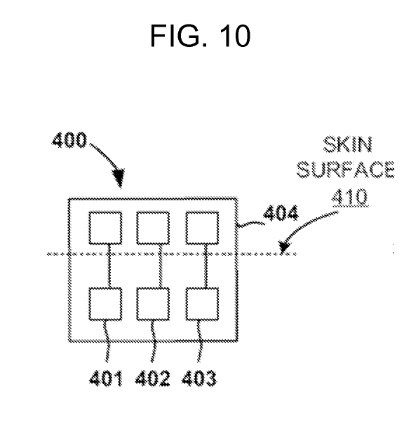

FIG. 10 schematically shows an embodiment of an analyte sensor 400 in accordance with the embodiments of the present disclosure. This sensor embodiment includes electrodes 401, 402 and 403 on a base 404. Electrodes (and/or other features) may be applied or otherwise processed using any suitable technology, e.g., chemical vapor deposition (CVD), physical vapor deposition, sputtering, reactive sputtering, printing, coating, ablating (e.g., laser ablation), painting, dip coating, etching, and the like. Materials include, but are not limited to, any one or more of aluminum, carbon (including graphite), cobalt, copper, gallium, gold, indium, iridium, iron, lead, magnesium, mercury (as an amalgam), nickel, niobium, osmium, palladium, platinum, rhenium, rhodium, selenium, silicon (e.g., doped polycrystalline silicon), silver, tantalum, tin, titanium, tungsten, uranium, vanadium, zinc, zirconium, mixtures thereof, and alloys, oxides, or metallic compounds of these elements.

The analyte sensor 400 may be wholly implantable in a user or may be configured so that only a portion is positioned within (internal) a user and another portion outside (external) a user. For example, the sensor 400 may include a first portion positionable above a surface of the skin 410, and a second portion positioned below the surface of the skin. In such embodiments, the external portion may include contacts (connected to respective electrodes of the second portion by traces) to connect to another device also external to the user such as a transmitter unit. While the embodiment of FIG. 10 shows three electrodes side-by-side on the same surface of base 404, other configurations are contemplated, e.g., fewer or greater electrodes, some or all electrodes on different surfaces of the base or present on another base, some or all electrodes stacked together, electrodes of differing materials and dimensions, etc.

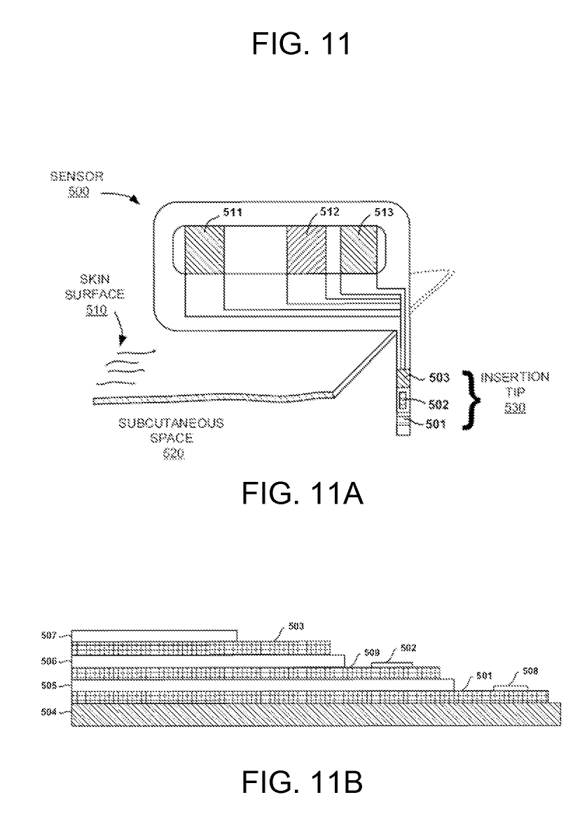

FIG. 11A shows a perspective view of an embodiment of an analyte sensor 500 having a first portion (which in this embodiment may be characterized as a major portion) positionable above a surface of the skin 510, and a second portion (which in this embodiment may be characterized as a minor portion) that includes an insertion tip 530 positionable below the surface of the skin, e.g., penetrating through the skin and into, e.g., the subcutaneous space 520, in contact with the user's biofluid, such as interstitial fluid. Contact portions of a working electrode 511, a reference electrode 512, and a counter electrode 513 are positioned on the first portion of the sensor 500 situated above the skin surface 510. A working electrode 501, a reference electrode 502, and a counter electrode 503 are shown at the second portion of the sensor 500 and particularly at the insertion tip 530. Traces may be provided from the electrodes at the tip to the contact, as shown in FIG. 11A. It is to be understood that greater or fewer electrodes may be provided on a sensor. For example, a sensor may include more than one working electrode and/or the counter and reference electrodes may be a single counter/reference electrode, etc.

FIG. 11B shows a cross sectional view of a portion of the sensor 500 of FIG. 11A. The electrodes 501, 502 and 503, of the sensor 500 as well as the substrate and the dielectric layers are provided in a layered configuration or construction. For example, as shown in FIG. 11B, in one embodiment, the sensor 500 (such as the analyte sensor unit 101 of FIG. 7), includes a substrate layer 504, and a first conducting layer 501 such as carbon, gold, etc., disposed on at least a portion of the substrate layer 504, and which may provide the working electrode. Also shown disposed on at least a portion of the first conducting layer 501 is a sensing element 508.

A first insulation layer 505, such as a first dielectric layer in certain embodiments, is disposed or layered on at least a portion of the first conducting layer 501, and further, a second conducting layer 509 may be disposed or stacked on top of at least a portion of the first insulation layer (or dielectric layer) 505. As shown in FIG. 11B, the second conducting layer 509 may provide the reference electrode 502, as described herein having an extended lifetime, which includes a layer of redox polymer as described herein.

A second insulation layer 506, such as a second dielectric layer in certain embodiments, may be disposed or layered on at least a portion of the second conducting layer 509. Further, a third conducting layer 503 may be disposed on at least a portion of the second insulation layer 506 and may provide the counter electrode 503. Finally, a third insulation layer 507 may be disposed or layered on at least a portion of the third conducting layer 503. In this manner, the sensor 500 may be layered such that at least a portion of each of the conducting layers is separated by a respective insulation layer (for example, a dielectric layer). The embodiments of FIGS. 11A and 11B show the layers having different lengths. In certain instances, some or all of the layers may have the same or different lengths and/or widths.

In certain embodiments, some or all of the electrodes 501, 502, 503 may be provided on the same side of the substrate 504 in the layered construction as described above, or alternatively, may be provided in a co-planar manner such that two or more electrodes may be positioned on the same plane (e.g., side-by side (e.g., parallel) or angled relative to each other) on the substrate 504. For example, co-planar electrodes may include a suitable spacing therebetween and/or include a dielectric material or insulation material disposed between the conducting layers/electrodes. Furthermore, in certain embodiments, one or more of the electrodes 501, 502, 503 may be disposed on opposing sides of the substrate 504. In such embodiments, contact pads may be one the same or different sides of the substrate. For example, an electrode may be on a first side and its respective contact may be on a second side, e.g., a trace connecting the electrode and the contact may traverse through the substrate.

As noted above, analyte sensors may include an analyte-responsive enzyme to provide a sensing element. Some analytes, such as oxygen, can be directly electrooxidized or electroreduced on a sensor, and more specifically at least on a working electrode of a sensor. Other analytes, such as glucose and lactate, require the presence of at least one electron transfer agent and/or at least one catalyst to facilitate the electrooxidation or electroreduction of the analyte. Catalysts may also be used for those analytes, such as oxygen, that can be directly electrooxidized or electroreduced on the working electrode. For these analytes, each working electrode includes a sensing element (see for example sensing element 508 of FIG. 11B) proximate to or on a surface of a working electrode. In many embodiments, a sensing element is formed near or on only a small portion of at least a working electrode.

Each sensing element includes one or more components constructed to facilitate the electrochemical oxidation or reduction of the analyte. The sensing element may include, for example, a catalyst to catalyze a reaction of the analyte and produce a response at the working electrode, an electron transfer agent to transfer electrons between the analyte and the working electrode (or other component), or both.

A variety of different sensing element configurations may be used. In certain embodiments, the sensing elements are deposited on the conductive material of a working electrode. The sensing elements may extend beyond the conductive material of the working electrode. In some cases, the sensing elements may also extend over other electrodes, e.g., over the counter electrode and/or reference electrode (or counter/reference is provided). In other embodiments, the sensing elements are contained on the working electrode, such that the sensing elements do not extend beyond the conductive material of the working electrode.

Sensing elements that are in direct contact with the working electrode may contain an electron transfer agent to transfer electrons directly or indirectly between the analyte and the working electrode, and/or a catalyst to facilitate a reaction of the analyte. For example, a glucose, lactate, or oxygen electrode may be formed having sensing elements which contain a catalyst, including glucose oxidase, glucose dehydrogenase, lactate oxidase, or laccase, respectively, and an electron transfer agent that facilitates the electrooxidation of the glucose, lactate, or oxygen, respectively.

In other embodiments the sensing elements are not deposited directly on the working electrode. Instead, the sensing elements 508 may be spaced apart from the working electrode, and separated from the working electrode, e.g., by a separation layer. A separation layer may include one or more membranes or films or a physical distance. In addition to separating the working electrode from the sensing elements, the separation layer may also act as a mass transport limiting layer and/or an interferent eliminating layer and/or a biocompatible layer.

In certain embodiments which include more than one working electrode, one or more of the working electrodes may not have corresponding sensing elements, or may have sensing elements that do not contain one or more components (e.g., an electron transfer agent and/or catalyst) needed to electrolyze the analyte. Thus, the signal at this working electrode may correspond to background signal which may be removed from the analyte signal obtained from one or more other working electrodes that are associated with fully-functional sensing elements by, for example, subtracting the signal.

In certain embodiments, the sensing elements include one or more electron transfer agents. Electron transfer agents that may be employed are electroreducible and electrooxidizable ions or molecules having redox potentials that are a few hundred millivolts above or below the redox potential of the standard calomel electrode (SCE). The electron transfer agent may be organic, organometallic, or inorganic. Examples of organic redox species are quinones and species that in their oxidized state have quinoid structures, such as Nile blue and indophenol. Examples of organometallic redox species are metallocenes including ferrocene. Examples of inorganic redox species are hexacyanoferrate (III), ruthenium hexamine, etc. Additional examples include those described in U.S. Pat. Nos. 6,736,957, 7,501,053 and 7,754,093, the disclosures of each of which are incorporated herein by reference in their entirety.

In certain embodiments, electron transfer agents have structures or charges which prevent or substantially reduce the diffusional loss of the electron transfer agent during the period of time that the sample is being analyzed. For example, electron transfer agents include but are not limited to a redox species, e.g., bound to a polymer which can in turn be disposed on or near the working electrode. The bond between the redox species and the polymer may be covalent, coordinative, or ionic. Although any organic, organometallic or inorganic redox species may be bound to a polymer and used as an electron transfer agent, in certain embodiments the redox species is a transition metal compound or complex, e.g., osmium, ruthenium, iron, and cobalt compounds or complexes. It will be recognized that many redox species described for use with a polymeric component may also be used, without a polymeric component.

Embodiments of polymeric electron transfer agents may contain a redox species covalently bound in a polymeric composition. An example of this type of mediator is poly(vinylferrocene). Another type of electron transfer agent contains an ionically-bound redox species. This type of mediator may include a charged polymer coupled to an oppositely charged redox species. Examples of this type of mediator include a negatively charged polymer coupled to a positively charged redox species such as an osmium or ruthenium polypyridyl cation. Another example of an ionically-bound mediator is a positively charged polymer including quaternized poly(4-vinyl pyridine) or poly(1-vinyl imidazole) coupled to a negatively charged redox species such as ferricyanide or ferrocyanide. In other embodiments, electron transfer agents include a redox species coordinatively bound to a polymer. For example, the mediator may be formed by coordination of an osmium or cobalt 2,2′-bipyridyl complex to poly(1-vinyl imidazole) or poly(4-vinyl pyridine).

Suitable electron transfer agents are osmium transition metal complexes with one or more ligands, each ligand having a nitrogen-containing heterocycle such as 2,2′-bipyridine, 1,10-phenanthroline, 1-methyl, 2-pyridyl biimidazole, or derivatives thereof. The electron transfer agents may also have one or more ligands covalently bound in a polymer, each ligand having at least one nitrogen-containing heterocycle, such as pyridine, imidazole, or derivatives thereof. One example of an electron transfer agent includes (a) a polymer or copolymer having pyridine or imidazole functional groups and (b) osmium cations complexed with two ligands, each ligand containing 2,2′-bipyridine, 1,10-phenanthroline, or derivatives thereof, the two ligands not necessarily being the same. Some derivatives of 2,2′-bipyridine for complexation with the osmium cation include but are not limited to 4,4′-dimethyl-2,2′-bipyridine and mono-, di-, and polyalkoxy-2,2′-bipyridines, including 4,4′-dimethoxy-2,2′-bipyridine. Derivatives of 1,10-phenanthroline for complexation with the osmium cation include but are not limited to 4,7-dimethyl-1,10-phenanthroline and mono, di-, and polyalkoxy-1,10-phenanthrolines, such as 4,7-dimethoxy-1,10-phenanthroline. Polymers for complexation with the osmium cation include but are not limited to polymers and copolymers of poly(1-vinyl imidazole) (referred to as “PVI”) and poly(4-vinyl pyridine) (referred to as “PVP”). Suitable copolymer substituents of poly(1-vinyl imidazole) include acrylonitrile, acrylamide, and substituted or quaternized N-vinyl imidazole, e.g., electron transfer agents with osmium complexed to a polymer or copolymer of poly(1-vinyl imidazole).

Embodiments may employ electron transfer agents having a redox potential ranging from about −200 mV to about +200 mV versus the standard calomel electrode (SCE). The sensing elements may also include a catalyst which is capable of catalyzing a reaction of the analyte. The catalyst may also, in some embodiments, act as an electron transfer agent. One example of a suitable catalyst is an enzyme which catalyzes a reaction of the analyte. For example, a catalyst, including a glucose oxidase, glucose dehydrogenase (e.g., pyrroloquinoline quinone (PQQ), dependent glucose dehydrogenase, flavine adenine dinucleotide (FAD) dependent glucose dehydrogenase, or nicotinamide adenine dinucleotide (NAD) dependent glucose dehydrogenase), may be used when the analyte of interest is glucose. A lactate oxidase or lactate dehydrogenase may be used when the analyte of interest is lactate. Laccase may be used when the analyte of interest is oxygen or when oxygen is generated or consumed in response to a reaction of the analyte.

In certain embodiments, a catalyst may be attached to a polymer, cross linking the catalyst with another electron transfer agent, which, as described above, may be polymeric. A second catalyst may also be used in certain embodiments. This second catalyst may be used to catalyze a reaction of a product compound resulting from the catalyzed reaction of the analyte. The second catalyst may operate with an electron transfer agent to electrolyze the product compound to generate a signal at the working electrode. Alternatively, a second catalyst may be provided in an interferent-eliminating layer to catalyze reactions that remove interferents.

In certain embodiments, the sensor works at a low oxidizing potential, e.g., a potential of about +40 mV vs. Ag/AgCl. This sensing elements use, for example, an osmium (Os)-based mediator constructed for low potential operation. Accordingly, in certain embodiments the sensing elements are redox active components that include: (1) osmium-based mediator molecules that include (bidente) ligands, and (2) glucose oxidase enzyme molecules. These two constituents are combined together in the sensing elements of the sensor.

A mass transport limiting layer (not shown), e.g., an analyte flux modulating layer, may be included with the sensor to act as a diffusion-limiting barrier to reduce the rate of mass transport of the analyte, for example, glucose or lactate, into the region around the working electrodes. The mass transport limiting layers are useful in limiting the flux of an analyte to a working electrode in an electrochemical sensor so that the sensor is linearly responsive over a large range of analyte concentrations and is easily calibrated. Mass transport limiting layers may include polymers and may be biocompatible. A mass transport limiting layer may provide many functions, e.g., biocompatibility and/or interferent-eliminating functions, etc.

In certain embodiments, a mass transport limiting layer is a membrane composed of crosslinked polymers containing heterocyclic nitrogen groups, such as polymers of polyvinylpyridine and polyvinylimidazole. Embodiments also include membranes that are made of a polyurethane, or polyether urethane, or chemically related material, or membranes that are made of silicone, and the like.

A membrane may be formed by crosslinking in situ a polymer, modified with a zwitterionic moiety, a non-pyridine copolymer component, and optionally another moiety that is either hydrophilic or hydrophobic, and/or has other desirable properties, in an alcohol-buffer solution. The modified polymer may be made from a precursor polymer containing heterocyclic nitrogen groups. For example, a precursor polymer may be polyvinylpyridine or polyvinylimidazole. Optionally, hydrophilic or hydrophobic modifiers may be used to “fine-tune” the permeability of the resulting membrane to an analyte of interest. Optional hydrophilic modifiers, such as poly(ethylene glycol), hydroxyl or polyhydroxyl modifiers, may be used to enhance the biocompatibility of the polymer or the resulting membrane.

A membrane may be formed in situ by applying an alcohol-buffer solution of a crosslinker and a modified polymer over the enzyme-containing sensing elements and allowing the solution to cure for about one to two days or other appropriate time period. The crosslinker-polymer solution may be applied over the sensing elements by placing a droplet or droplets of the membrane solution on the sensor, by dipping the sensor into the membrane solution, by spraying the membrane solution on the sensor, and the like. Generally, the thickness of the membrane is controlled by the concentration of the membrane solution, by the number of droplets of the membrane solution applied, by the number of times the sensor is dipped in the membrane solution, by the volume of membrane solution sprayed on the sensor, or by any combination of these factors. A membrane applied in this manner may have any combination of the following functions: (1) mass transport limitation, i.e., reduction of the flux of analyte that can reach the sensing elements, (2) biocompatibility enhancement, or (3) interferent reduction.

In some instances, the membrane may form one or more bonds with the sensing elements. By bonds is meant any type of an interaction between atoms or molecules that allows chemical compounds to form associations with each other, such as, but not limited to, covalent bonds, ionic bonds, dipole-dipole interactions, hydrogen bonds, London dispersion forces, and the like. For example, in situ polymerization of the membrane can form crosslinks between the polymers of the membrane and the polymers in the sensing elements. In certain embodiments, crosslinking of the membrane to the sensing element facilitates a reduction in the occurrence of delamination of the membrane from the sensor.

In certain embodiments, the sensing system detects hydrogen peroxide to infer glucose levels. For example, a hydrogen peroxide-detecting sensor may be constructed in which the sensing elements include an enzyme such as glucose oxidase, glucose dehydrogenase, or the like, and is positioned on the working electrode. The sensing elements may be covered by one or more layers, e.g., a membrane that is selectively permeable to glucose. Once the glucose passes through the membrane, it is oxidized by the enzyme and reduced glucose oxidase can then be oxidized by reacting with molecular oxygen to produce hydrogen peroxide.

Certain embodiments include a hydrogen peroxide-detecting sensor constructed from sensing elements prepared by combining together, for example: (1) a redox mediator having a transition metal complex including an Os polypyridyl complex with oxidation potentials of about +200 mV vs. SCE, and (2) periodate oxidized horseradish peroxidase (HRP). Such a sensor functions in a reductive mode; the working electrode is controlled at a potential negative to that of the Os complex, resulting in mediated reduction of hydrogen peroxide through the HRP catalyst.

In another example, a potentiometric sensor can be constructed as follows. Glucose-sensing elements may be constructed by combining together (1) a redox mediator having a transition metal complex including Os polypyridyl complexes with oxidation potentials from about −200 mV to +200 mV vs. SCE, and (2) glucose oxidase. This sensor can then be used in a potentiometric mode, by exposing the sensor to a glucose containing solution, under conditions of zero current flow, and allowing the ratio of reduced/oxidized Os to reach an equilibrium value. The reduced/oxidized Os ratio varies in a reproducible way with the glucose concentration, and will cause the electrode's potential to vary in a similar way.

The substrate may be formed using a variety of non-conducting materials, including, for example, polymeric or plastic materials and ceramic materials. Suitable materials for a particular sensor may be determined, at least in part, based on the desired use of the sensor and properties of the materials.

In some embodiments, the substrate is flexible. For example, if the sensor is configured for implantation into a user, then the sensor may be made flexible (although rigid sensors may also be used for implantable sensors) to reduce pain to the user and damage to the tissue caused by the implantation of and/or the wearing of the sensor. A flexible substrate often increases the user's comfort and allows a wider range of activities. Suitable materials for a flexible substrate include, for example, non-conducting plastic or polymeric materials and other non-conducting, flexible, deformable materials. Examples of useful plastic or polymeric materials include thermoplastics such as polycarbonates, polyesters (e.g., Mylar™ and polyethylene terephthalate (PET)), polyvinyl chloride (PVC), polyurethanes, polyethers, polyamides, polyimides, or copolymers of these thermoplastics, such as PETG (glycol-modified polyethylene terephthalate).

In other embodiments, the sensors are made using a relatively rigid substrate to, for example, provide structural support against bending or breaking. Examples of rigid materials that may be used as the substrate include poorly conducting ceramics, such as aluminum oxide and silicon dioxide. An implantable sensor having a rigid substrate may have a sharp point and/or a sharp edge to aid in implantation of a sensor without an additional insertion device.

It will be appreciated that for many sensors and sensor applications, both rigid and flexible sensors will operate adequately. The flexibility of the sensor may also be controlled and varied along a continuum by changing, for example, the composition and/or thickness of the substrate.

In addition to considerations regarding flexibility, it is often desirable that implantable sensors should have a substrate which is physiologically harmless, for example, a substrate approved by a regulatory agency or private institution for in vivo use.

The sensor may include optional features to facilitate insertion of an implantable sensor. For example, the sensor may be pointed at the tip to ease insertion. In addition, the sensor may include a barb which assists in anchoring the sensor within the tissue of the user during operation of the sensor. However, the barb is typically small enough so that little damage is caused to the subcutaneous tissue when the sensor is removed for replacement.

An implantable sensor may also, optionally, have an anticlotting agent disposed on a portion of the substrate which is implanted into a user. This anticlotting agent may reduce or eliminate the clotting of blood or other body fluid around the sensor, particularly after insertion of the sensor. Blood clots may foul the sensor or irreproducibly reduce the amount of analyte which diffuses into the sensor. Examples of useful anticlotting agents include heparin and tissue plasminogen activator (TPA), as well as other known anticlotting agents.

The anticlotting agent may be applied to at least a portion of that part of the sensor that is to be implanted. The anticlotting agent may be applied, for example, by bath, spraying, brushing, or dipping, etc. The anticlotting agent is allowed to dry on the sensor. The anticlotting agent may be immobilized on the surface of the sensor or it may be allowed to diffuse away from the sensor surface. The quantities of anticlotting agent disposed on the sensor may be below the amounts typically used for treatment of medical conditions involving blood clots and, therefore, have only a limited, localized effect.

The following examples are offered by way of illustration and not by way of limitation.

### EXPERIMENTAL

### Example 1

### Interstitial Fluid Collection

Interstitial fluid from eight human subjects was collected using the method and device as shown in FIG. 2. First, a forearm region of each subject was located, and then a skin stabilizing frame was attached such that the frame surrounded the region. To abrade the stratum corneum, a DREMEL™ rotary device was used. The rotary device included a bit with a grindstone tip and a housing to prevent the grindstone tip from abrading the skin beyond a depth of either 1.5 or 2 mm. Prior to abrading the skin, the rotary device was mated with the skin stabilizing frame, the frame having notches complementary to bumps on the housing of the rotary device. Each subject was abraded at two regions on the same forearm, with three sites abraded at each region. Each set of three sites was designated a “well”. Accordingly, two wells for each subject were generated.

Results for each well of each subject are provided in Table 1. Each site was abraded for either 20 or 40 seconds. The speed of the rotary device was either between 5,000-6,000 rpm (speed “3”) or between 6,001 and 10,000 rpm (speed “4”). The three sites at each well were abraded to a depth of either 1.5 mm or 2 mm. Once a well of three abrasion sites was generated, an EZVAC™ vacuum device placed over the well, and a vacuum pressure was applied to the well for either 1 or 3 minutes. The amount of interstitial fluid extracted from each well ranged from 0.0 μL to 5.5 μL. Also measured was the length of time that each well yielded interstitial fluid.

TABLE 1
INTERSTITIAL FLUID (ISF) COLLECTION DATA

| Sub- | | Abrasion | | | Vacuum | | |
|---|---|---|---|---|---|---|---|
| ject | | Time | | Depth | Time | Amount | Length |
| No. | Well | (sec) | Speed | (mm) | (min) | (μL) | (hours) |
| 201 | 1 | 40 | 3 | 2 | 1 | 0.0 | 0.0 |
| 201 | 2 | 40 | 4 | 1.5 | 1 | 3.0 | 1.25 |
| 202 | 1 | 40 | 3 | 1.5 | 3 | 0.0 | 0.0 |
| 202 | 2 | 40 | 4 | 2 | 3 | 0.0 | 0.0 |
| 203 | 1 | 20 | 3 | 2 | 3 | 5.5 | 9 |
| 203 | 2 | 20 | 4 | 1.5 | 3 | 5.5 | 19 |
| 204 | 1 | 20 | 3 | 1.5 | 1 | 1.5 | 0 |
| 204 | 2 | 20 | 4 | 2 | 1 | 2.0 | 1 |
| 205 | 1 | 20 | 4 | 2 | 1 | 0.0 | 0 |
| 205 | 2 | 20 | 3 | 1.5 | 1 | 3.0 | 9 |
| 206 | 1 | 40 | 4 | 1.5 | 1 | 3.0 | 2 |
| 206 | 2 | 40 | 3 | 2 | 1 | 0.0 | 0 |
| 207 | 1 | 20 | 4 | 1.5 | 3 | 4.5 | 6 |
| 207 | 2 | 20 | 3 | 2 | 3 | 4.5 | 6 |
| 208 | 1 | 40 | 4 | 2 | 3 | 3.0 | 1 |
| 208 | 2 | 40 | 3 | 1.5 | 3 | 1.0 | 0 |

### Example 2

### Measurement of Interstitial Fluid Glucose Concentration by Ion Chromatography

The present example provides a protocol for measuring glucose concentration in an interstitial fluid sample by ion chromatography.

Background

NERL glucose standards (50, 100, 200, 400 mg/dL) are used as standards in this method since there is no commercially available glucose standard in interstitial fluid. In the Ion Chromatography method described in this document, the samples and standards are diluted similarly prior to Ion Chromatography. Therefore, the additional components present in the interstitial fluid analytical samples compared to the glucose standards are not expected to have any impact on the analysis.

The following definitions are used in this protocol: IC (ion chromatography); ISF (interstitial fluid); RSD (relative standard deviation); and PES (polyethersulfone).

Equipment

The following equipment was used during the procedure: ion chromatography system (Dionex ICS3000 Ion Chromatography System with Helium degasser); column (Dionex CarboPac PA10, 2 mm diameter); Detector (Dionex ED electrochemical detector); Pipetman, various sizes (BioHit); centrifuge tubes (Eppendorf 5415C centrifuge tubes); rack (VWR microfuge rack); nutator (VWR nutator).

Materials

The following materials were used during the procedure: clinical samples (Abbott Diabetes Care); NERL glucose standards (50, 100, 200, 400 mg/dL) (ThermoFisher); 50% sodium hydroxide solution (VWR); deionized water, obtained from Barnstead E pure Model D5023 (VWR); benzoic acid, 99.5% (Aldrich); BINDPRO™ (Biotech Support Group); PES 0.22 um solvent filtration apparatus, disposable (VWR); Autosampler vials, 1.8 mL, glass, slitted screw cap (Dionex); 0.3 mL, glass, slitted screw cap (Dionex); 1.5 mL microfuge tubes (VWR); 0.6 mL microfuge tubes (VWR); pipet tips, various sizes (VWR); 0.2 μm centrifugal filtration devices (VWR).

1—Mobile Phase Preparation

The dilution volumes may be modified provided that the respective concentrations remain the same as stated in the section below. 200 mM NaOH is prepared by transferring 16.0 g of 50% sodium hydroxide solution into a 1 L volumetric flask and diluting to the mark with DI water (solid sodium hydroxide should not be used, as the solid tends to absorb CO2 from the air and thus causes problems with the baseline in the IC; the IC instrument is set up such that the 200 mM NaOH solution is blanketed with helium during storage and analysis, which also serves to prevent CO2 contamination). Mix by inverting flask 5 times. Filter through a 0.22 μm PES (polyethersulfone) vacuum filter. Degas the mobile phase for at least two (2) hours with helium prior to initiating analysis.

2—Standard Preparation

The dilution volumes for the standards below may be modified provided that the respective concentrations remain the same as stated in the sections below. The volumes for each standard below results in a 500-fold dilution of each of the commercial NERL glucose standards. However, it should be noted that 500 uL is the maximum volume for a 0.2 μm centrifugal filter used in the filtration step above and others below.

Standard Solution 1 (100 μg/dL NERL D-Glucose Solution)

Accurately transfer 100 μL of 50 mg/dL NERL D-Glucose standard solution into a 50 mL volumetric flask. Dilute to the mark with DI water. Mix well by inverting the flask 5 times. Accurately transfer 250 μL to a 0.6 mL microfuge tube. Add 5 μL of BINDPRO™ suspension into the tube using a wide-bore or cut-off pipet tip. Wipe off excess BINDPRO™ from the pipet tip with a KIMWIPE® prior to adding. Place the tube on a nutator for 20 minutes until it is fully suspended. Filter the mixture using a 0.2 centrifugal filter and transfer the filtrate into a 0.3 mL plastic autosampler vial. Prepare duplicate Standard Solution 1.

Standard Solution 2 (200 μg/dL NERL D-Glucose Solution)

Accurately transfer 100 μL of 100 mg/dL NERL D-Glucose standard solution into a 50 mL volumetric flask. Dilute to the mark with DI water. Mix well by inverting the flask 5 times. Accurately transfer 250 μL to a 0.6 mL microfuge tube. Add 5 μL of BINDPRO™ suspension into the tube using a wide-bore or cut-off pipet tip. Wipe off excess BINDPRO™ from the pipet tip with a KIMWIPE® prior to adding. Place the tube on a nutator for 20 minutes until it is fully suspended. Filter the mixture using a 0.2 μm centrifugal filter and transfer the filtrate into a 0.3 mL plastic autosampler vial. Prepare duplicate Standard Solution 2.

Standard Solution 3 (400 μg/dL NERL D-Glucose Solution)

Accurately transfer 100 μL of 200 mg/dL NERL D-Glucose standard solution into a 50 mL volumetric flask. Dilute to the mark with DI water. Mix well by inverting the flask 5 times. Accurately transfer 250 μL to a 0.6 mL microfuge tube. Add 5 μL of BINDPRO™ suspension into the tube using a wide-bore or cut-off pipet tip. Wipe off excess BINDPRO™ from the pipet tip with a KIMWIPE® prior to adding. Place the tube on a nutator for 20 minutes until it is fully suspended. Filter the mixture using a 0.2 centrifugal filter and transfer the filtrate into a 0.3 mL plastic autosampler vial. Prepare duplicate Standard Solution 3.

Standard Solution 4 (800 μg/dL NERL D-Glucose Solution)

Accurately transfer 100 μL of 400 mg/dL NERL D-Glucose standard solution into a 50 mL volumetric flask. Dilute to the mark with DI water. Mix well by inverting the flask 5 times. Accurately transfer 250 μL to a 0.6 mL microfuge tube. Add 5 μL of BINDPRO™ suspension into the tube using a wide-bore or cut-off pipet tip. Wipe off excess BINDPRO™ from the pipet tip with a KIMWIPE® prior to adding. Place the tube on a nutator for 20 minutes until it is fully suspended. Filter the mixture using a 0.2 centrifugal filter and transfer the filtrate into a 0.3 mL plastic autosampler vial. Prepare duplicate Standard Solution 4.

3—Clinical Sample Preparation

The clinical samples consist of 0.5 μL of human interstitial fluid (ISF) in 250 μL of aqueous benzoic acid solution. The clinical samples are immediately frozen and remain frozen until just before IC analysis.

Clinical samples are thawed and prepared for analysis as follows. Accurately transfer 125 μL of a thawed clinical sample into a 0.6 mL microfuge tube. Add 2.5 μL BINDPRO™ solution into the tube. Place the tube on a nutator for 20 minutes. Filter the sample using a 0.2 μm centrifugal filter and transfer the filtrate into a 0.3 mL plastic autosampler vial. Prepare an additional analytical sample as described above and store in the freezer for later use, if needed.

4—Ion Chromatography Conditions

The following conditions were used for ion chromatography: the flow rate was 0.20 mL per minute; the injection volume was 10 μL; data acquisition was 18 minutes; equilibration time was 3.5 hr prior to first run; no equilibration between runs; mobile phase composition (isocratic) (DI Water: 200 mM NaOH=90:10).

Typical Injection Sequence for Sample Assay

A typical injection sequence when analyzing interstitial fluid samples is provided in Table 2.

TABLE 2
ION CHROMATOGRAPHY INJECTION SEQUENCE

| Injection | Sample Identity |
|---|---|
| 1 | blank (water) |
| 2 | benzoic acid solution |
| 3 | BindPro solution |
| 4 | Standard 1 |
| 5 | Standard 1 |
| 6 | Standard 1 |
| 7 | Standard 1 |
| 8 | Standard 1 |
| 9 | Standard 1 |
| 10 | Standard 2 |
| 11 | Standard 3 |
| 12 | Standard 4 |
| 13 | Sample 1-1 |
| 14 | Sample 1-2 |
| 15 | Sample 2-1 |
| 16 | Sample 2-2 |
| 17 | Sample 3-1 |
| 18 | Sample 3-2 |
| 19 | Sample 4-1 |
| 20 | Sample 4-2 |
| 21 | Sample 5-1 |
| 22 | Sample 5-2 |
| 23 | Sample 6-1 |
| 24 | Sample 6-2 |

The above sequence is recommended for a sample against a reference standard. If additional samples are injected, a standard should be included for every six sample injections.

System Suitability

System suitability is tested using Standard 1. The percent relative Standard Deviation (% RSD) of the six replicate injections of Standard 1 should be less than or equal to 3%. Efficiency (N): the theoretical plates for the glucose peak should be ≧3000.

5—Analysis

The assay method used to determine the concentration of glucose in samples is by comparison with the NERL D-Glucose standard of known concentration. The analysis steps are as follows.

Inject the six system suitability Standard 1 solutions (six total injections). After the six system suitability injections have been made, inject the remaining standards once each. The last system suitability standard 1 should be included in the calibration curve. All clinical samples are injected twice. The average of the two sample injections will be used for a single reportable value. If the percent difference between two injections is greater than 10%, the data is considered invalid. Determine the peak area of the standard and sample from their respective chromatograms. Determine the calibration curve by linear regression (peak area vs. standard concentration). All standard injections other than the six system suitability standard injections should be used for the calibration curve. Calculate the glucose concentration in the samples based on the calibration curve.

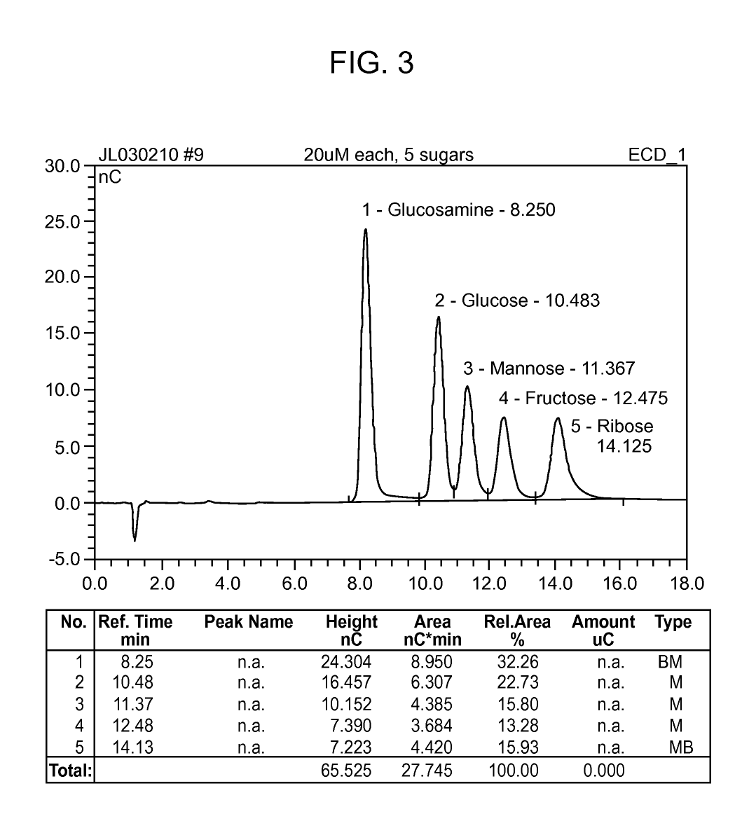

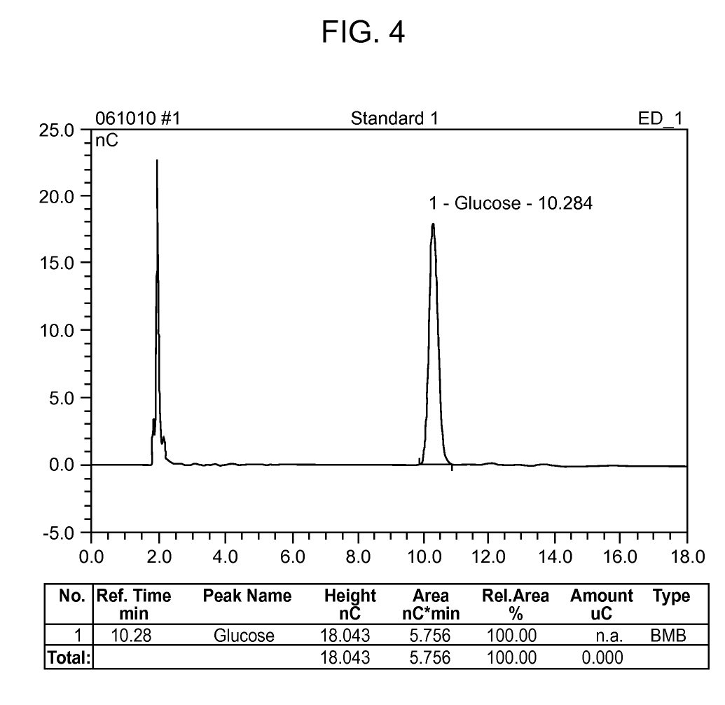

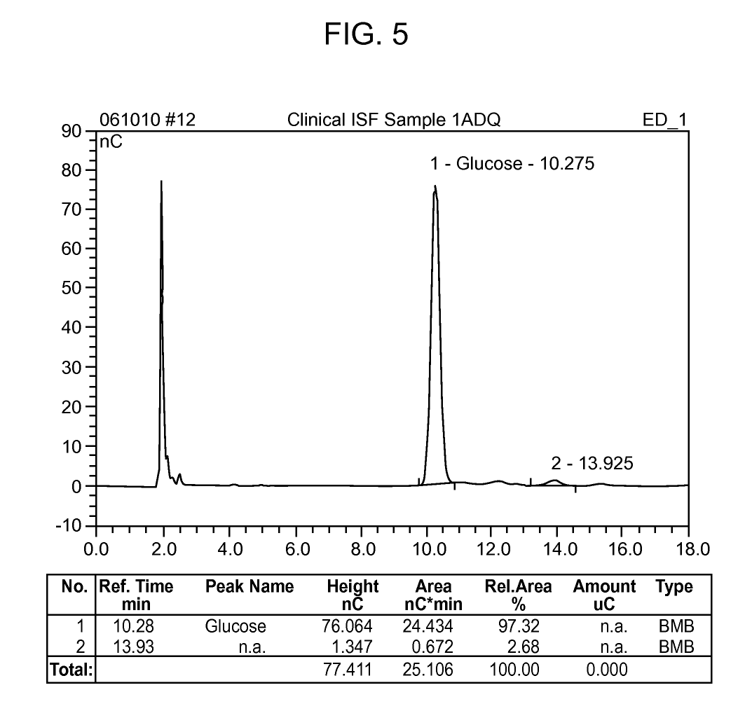

Ion chromatography results for a typical example sugar test mixture are shown in FIG. 3. From left to right, peaks for glucosamine, glucose, mannose, fructose and ribose are distinguishable. A typical example of the NERL D-glucose standard is shown in FIG. 4, with the glucose peak labeled “1” and indicating a residence time of 10.28 minutes in this example. A typical example of a human interstitial fluid sample is shown in FIG. 5, with the glucose peak labeled “1” and indicating a residence time of 10.28 minutes.

### Example 3

### Assessing the Accuracy of a Continuous Glucose Monitor

A number of approaches have been developed to statistically assess the accuracy of self-monitoring of blood glucose systems/monitors. A widely accepted approach is the Clarke Error Grid analysis, which describes the clinical accuracy of self-monitoring blood of glucose systems/monitors over the entire range of blood glucose values. The analysis takes into account the absolute value of the glucose concentration generated by the system, the absolute value of the reference blood glucose value, the relative difference between the two values, and the clinical significance of the difference. See Clarke, et al., “Evaluating Clinical Accuracy of Systems for Self-Monitoring of Blood Glucose” Diabetes Care 10(5): 622-628 (1987).

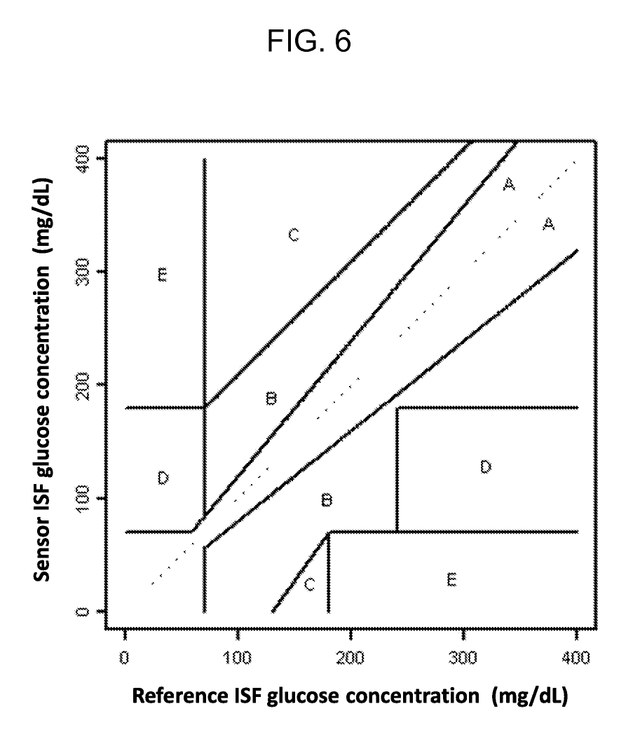

The Clarke error grid is a Cartesian diagram in which the glucose concentration values from the sensor/monitor being tested are displayed on the y-axis, while the values obtained using the reference method are displayed on the x-axis. See FIG. 6. The diagonal (dashed diagonal line in FIG. 6) represents perfect agreement between the two values. The readings of a glucose monitor are compared to a laboratory method on a scattergram and the graph is divided into zones that represent the error as it would relate to therapy. Points below and above the line indicate overestimation and underestimation, respectively, by the monitor. Data falling within Zone A are clinically accurate and deviate from the reference by no more than 20% (or are in the hypoglycemic range). Data falling within Zone B deviate from the reference by more than 20%, but are considered clinically acceptable. Values in Zone C would lead to unnecessary corrective treatment. Data falling within Zone D result in dangerous failure to detect hypo- or hyperglycemia, while data falling within Zone E would lead to erroneous treatment of hypo- or hyper-glycemia.

In this example, each subject wears a continuous glucose monitor on an arm or abdomen, the sensor of the monitor being subcutaneously implanted to generate an interstitial fluid glucose concentration measurement. During, shortly before, or shortly after the measurement is taken by the monitor, a reference interstitial fluid sample is obtained from a region of the subject in close proximity to the monitor. The reference interstitial fluid sample is obtained using the abrading, vacuum and collection procedure as described herein (e.g., see the section entitled “Methods of Measuring the Concentration of Analytes in Interstitial Fluid Samples” and FIG. 2). The glucose concentration of the reference interstitial fluid sample is determined by ion chromatography, e.g., as described in Example 2. Optionally, the glucose concentration of the reference interstitial fluid sample is normalized to the concentration of a reference analyte, such as a physiological salt component.

The above procedure is performed on a plurality of subjects. For each subject, the interstitial fluid glucose concentration value obtained from continuous glucose monitor is compared to the reference interstitial fluid glucose concentration as determined by ion chromatography. Collected data may be plotted on a Clarke Error Grid as described above, and as shown in FIG. 6.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of measuring the concentration of an analyte in an interstitial fluid sample, the method comprising:
abrading an epidermal skin layer of a subject at a first site;
applying a vacuum to the abraded epidermal skin layer such that interstitial fluid presents on a surface of the abraded epidermal skin layer;
collecting the presented interstitial fluid to obtain the interstitial fluid sample; and
measuring the concentration of the analyte in the interstitial fluid sample by ion chromatography.

2. The method according to claim 1, further comprising normalizing the analyte concentration to the concentration of a reference analyte in the interstitial fluid sample.

3. The method according to claim 2, wherein the reference analyte is a physiological salt component.

4. The method according to claim 3, wherein the physiological salt component is selected from the group consisting of: sodium, potassium, magnesium, calcium, chloride, hydrogen phosphate, hydrogen carbonate, and combinations thereof.

5. The method according to claim 1, wherein the abrading is performed using a rotary device.

6. The method according to claim 5, wherein the rotary device comprises a rotatable grindstone.

7. The method according to claim 6, wherein the rotary device comprises a housing that prevents the rotatable grindstone from abrading a blood vessel.

8. The method according to claim 1, wherein the abrading comprises abrading the epidermal skin layer at a depth ranging from about 0.5 to 3 mm.

9. The method according to claim 1, wherein the abrading comprises abrading the stratum corneum.

10. The method according to claim 1, wherein the first site is located at an area of the subject selected from the group consisting of: a forearm, an upper arm, and an abdomen.

11. The method according to claim 1, wherein the abrading further comprises abrading an epidermal skin layer of the subject at a second site.

12. The method according to claim 11, wherein the second site is adjacent to the first site.

13. The method according to claim 11, wherein the second site is contiguous with the first site.

14. The method according to claim 1, wherein applying a vacuum comprises applying a vacuum pressure that does not cause blood to present on the abraded epidermal skin layer.

15. The method according to claim 1, wherein applying a vacuum comprises applying a vacuum pressure ranging from about 6 to 20 inches of mercury.

16. The method according to claim 1, wherein the collecting is performed using a micro-capillary tube.

17. The method according to claim 1, further comprising diluting the interstitial fluid sample prior to measuring the concentration of the analyte.

18. The method according to claim 1, wherein the analyte is glucose.

19. A method of determining accuracy of a subcutaneously implanted analyte sensor, the method comprising:
determining an interstitial fluid analyte concentration from a subject using the subcutaneously implanted analyte sensor;
determining a reference interstitial fluid analyte concentration from the subject, wherein the determining comprises:
abrading an epidermal skin layer of the subject at a first site;
applying a vacuum to the abraded epidermal skin layer such that interstitial fluid presents on a surface of the abraded epidermal skin layer; and
measuring the concentration of the analyte in the presented interstitial fluid; and
comparing the interstitial fluid analyte concentration from the subcutaneously implanted analyte sensor to the reference interstitial fluid analyte concentration, thereby determining the accuracy of the subcutaneously implanted analyte sensor.

20. The method according to claim 19, further comprising normalizing the reference interstitial fluid analyte concentration to the concentration of a physiological salt component in the presented interstitial fluid.

21. The method according to claim 20, wherein the physiological salt component is selected from the group consisting of: sodium, potassium, magnesium, calcium, chloride, hydrogen phosphate, hydrogen carbonate, and combinations thereof.

22. The method according to claim 19, wherein the abrading is performed using a rotary device.

23. The method according to claim 22, wherein the rotary device comprises a rotatable grindstone.

24. The method according to claim 23, wherein the rotary device comprises a housing that prevents the rotatable grindstone from abrading a blood vessel.

25. The method according to claim 19, wherein the abrading comprises abrading the epidermal skin layer at a depth ranging from about 0.5 to 3 mm.

26. The method according to claim 19, wherein the abrading comprises abrading the stratum corneum.

27. The method according to claim 19, wherein the first site is located at an area of the subject selected from the group consisting of: a forearm, an upper arm, and an abdomen.

28. The method according to claim 19, wherein the abrading further comprises abrading an epidermal skin layer of the subject at a second site.

29. The method according to claim 28, wherein the second site is adjacent to the first site.

30. The method according to claim 28, wherein the second site is contiguous with the first site.

31. The method according to claim 19, wherein applying a vacuum pressure comprises applying a pressure that does not cause blood to present on the abraded epidermal skin layer.

32. The method according to claim 19, wherein applying a vacuum to the abraded epidermal skin layer comprises applying a vacuum pressure ranging from about 6 to 20 inches of mercury.

33. The method according to claim 19, further comprising diluting the presented interstitial fluid prior to measuring the concentration of the analyte in the presented interstitial fluid.

34. The method according to claim 19, wherein measuring the concentration of the analyte in the presented interstitial fluid comprises measuring the concentration of the analyte by ion chromatography.

35. The method according to claim 19, wherein the analyte is glucose.

36. A method of determining a calibration factor for a subcutaneously implanted analyte sensor, the method comprising:
determining an interstitial fluid analyte concentration from a subject using the subcutaneously implanted analyte sensor;
determining a reference interstitial fluid analyte concentration from the subject, wherein the determining comprises:
abrading an epidermal skin layer of the subject at a first site;
applying a vacuum to the abraded epidermal skin layer such that interstitial fluid presents on a surface of the abraded epidermal skin layer; and
measuring the concentration of the analyte in the presented interstitial fluid;
comparing the interstitial fluid analyte concentration from the subcutaneously implanted analyte sensor to the reference interstitial fluid analyte concentration to determine a difference between the concentrations; and
determining the calibration factor for the subcutaneously implanted analyte sensor based on the difference.

37. The method according to claim 36, further comprising normalizing the reference interstitial fluid analyte concentration to the concentration of a physiological salt component in the presented interstitial fluid.

38. The method according to claim 37, wherein the physiological salt component is selected from the group consisting of: sodium, potassium, magnesium, calcium, chloride, hydrogen phosphate, hydrogen carbonate, and combinations thereof.

39. The method according to claim 36, wherein the abrading is performed using a rotary device.

40. The method according to claim 39, wherein the rotary device comprises a rotatable grindstone.

41. The method according to claim 40, wherein the rotary device comprises a housing that prevents the rotatable grindstone from abrading a blood vessel.

42. The method according to claim 36, wherein the abrading comprises abrading the epidermal skin layer at a depth ranging from about 0.5 to 3 mm.

43. The method according to claim 36, wherein the abrading comprises abrading the stratum corneum.

44. The method according to claim 36, wherein the first site is located at an area of the subject selected from the group consisting of: a forearm, an upper arm, and an abdomen.

45. The method according to claim 36, wherein the abrading further comprises abrading an epidermal skin layer of the subject at a second site.

46. The method according to claim 45, wherein the second site is adjacent to the first site.

47. The method according to claim 45, wherein the second site is contiguous with the first site.

48. The method according to claim 36, wherein applying a vacuum comprises applying a vacuum pressure that does not cause blood to present on the abraded epidermal skin layer.

49. The method according to claim 36, wherein applying a vacuum comprises applying a vacuum pressure ranging from about 6 to 20 inches of mercury.

50. The method according to claim 36, further comprising diluting the presented interstitial fluid prior to measuring the concentration of the analyte in the presented interstitial fluid.

51. The method according to claim 36, wherein measuring the concentration of the analyte in the presented interstitial fluid comprises measuring the concentration of the analyte by ion chromatography.

52. The method according to claim 36, wherein the analyte is glucose.

53. A method of determining the concentration of an analyte in a bodily sample, the method comprising:
measuring the concentration of an analyte in the sample;
measuring the concentration of a physiological salt component in the sample; and
normalizing the analyte concentration based on the concentration of the physiological salt component, thereby determining the concentration of the analyte in the bodily sample.

54. The method according to claim 53, wherein the bodily sample is an interstitial fluid sample.

55. The method according to claim 54, further comprising obtaining the interstitial fluid sample, wherein the obtaining comprises:
abrading an epidermal skin layer of a subject;
applying a vacuum to the abraded epidermal skin layer such that the interstitial fluid sample presents on a surface of the abraded epidermal skin layer; and
collecting the interstitial fluid sample presented on the surface.

56. The method according to claim 53, wherein measuring the concentration of an analyte in the sample comprises measuring the concentration of the analyte by ion chromatography.

57. The method according to claim 53, wherein the analyte is glucose.

58. The method according to claim 53, wherein the physiological salt component is selected from the group consisting of: sodium, potassium, magnesium, calcium, chloride, hydrogen phosphate, hydrogen carbonate, and combinations thereof.

59-91. (canceled)
